(19) United States
(12) Patent Application Publication
(10) Pub. No.: US 2021/0244768 A1
(43) Pub. Date: Aug. 12, 2021

(54) CELL COMPOSITIONS AND USES THEREOF

(71) Applicant: THE UNIVERSITY OF SYDNEY, SYDNEY, NEW SOUTH WALES (AU)

(72) Inventors: GREG NEELY, SYDNEY, NEW SOUTH WALES (AU); LESLIE CARON, SYDNEY, NEW SOUTH WALES (AU); JOHN MANION, SYDNEY, NEW SOUTH WALES (AU)

(21) Appl. No.: 16/972,926

(22) PCT Filed: Jun. 7, 2019
(86) PCT No.: PCT/AU2019/050594

§ 371 Date: Dec. 7, 2020

(30) Foreign Application Priority Data
Jun. 8, 2018 (AU) 2018902072

(51) Int. Cl. A61K 35/30; C12N 5/0793; A61K 38/18; A61K 31/221; A61K 31/497; A61K 9/00; A61P 25/02

(52) U.S. Cl. CPC A61K 35/30; C12N 5/0619; A61K 38/185; A61K 31/221; A61K 31/497; A61K 9/0085; C12N 2501/119; C12N 2506/45; C12N 2501/13; C12N 2501/845; C12N 2501/155; C12N 2501/415; C12N 2501/41; A61P 25/02

(57) ABSTRACT

This invention relates to compositions and methods for the transplantation of GABAergic neurons. GABAergic neurons and compositions comprising the same according to the present invention may be used as cell-based therapies for restoring or reinforcing central inhibition in the nervous system of a subject and for the treatment of neurological conditions, diseases and disorders associated with impaired or aberrant neural function. In a preferred embodiment, the transplant composition comprise of GABAergic neurons, a GFR-alpha agonist, and at least one cell death inhibitor, and that the GABAergic neurons are generated by differentiating pluripotent stem cells, multipotent stem cells, or progenitor cells.

### CROSS REFERENCE TO RELATED APPLICATIONS

The present application claims priority to Australian Provisional Application No. 2018902072, filed 8 Jun. 2018, the entire content of which is incorporated herein by cross-reference.

This invention relates to compositions and methods for the transplantation of GABAergic neurons. GABAergic neurons and compositions comprising the same according to the present invention may be used in cell-based therapies for restoring or reinforcing central inhibition in the nervous system of a subject and for the treatment neurological conditions, diseases and disorders associated with impaired or aberrant neural function.

### BACKGROUND

Chronic pain has an enormous impact on the quality of life for billions of patients, families, and caregivers worldwide, and current therapies do not adequately address pain for most patients. Globally, chronic pain is estimated to cost trillions of dollars per year, similar to the cost of cancer, heart disease, or diabetes. Importantly, lack of effective treatments for chronic pain has had knock-on effects in our society, for example the opioid epidemic, where since 2000, >200,000 people have died from prescription opioid overdoses. Importantly, the incidence of chronic pain increases with age and associates with many age-related diseases such as cancer and diabetes; thus, in an aging society chronic pain represents a clear and unmet clinical issue. Neuropathic pain can manifest as burning, stabbing, and stinging pain that is most similar in quality to electric shock.

Neuropathic pain (e.g. sciatica, back pain, cancer pain, diabetic pain, accidental injury) is generally refractory to available therapies, with front line anti-neuropathics providing adequate pain relief for only ˜25% of patients. Treatment with morphine may provide some pain relief or distraction in acute settings, but a chronic morphine regime leads to issues with addiction and tolerance that cannot be ignored. There has been some success with non-opiates such as pregabalin and nortriptyline, but these drugs have varying efficacies and do not adequately address pain intensity.

Despite decades of research into the molecular and physiological mechanisms that contribute to neuropathic pain, it is still not completely clear what should be targeted to treat the underlying pathology responsible for neuropathic pain. Better, non-addictive pain therapies that can reverse, or even resolve chronic disease are required.

Furthermore, the provision of cell-based therapies for the restoring or reinforcing central inhibition in the nervous system of a subject and for the treatment neurological conditions, diseases and disorders associated with impaired or inadequate inhibitory interneuron activity or increased excitatory neuron function represents an unmet need.

### SUMMARY OF INVENTION

Numbered statements of the invention are as follows:
1. A transplant composition for administration to a mammal, said transplant composition comprising a population of GABAergic neurons, a GFRalpha agonist, an apoptosis inhibitor, and a necrosis inhibitor, wherein said GABAergic neurons are generated by differentiating pluripotent stem cells, or multipotent stem or progenitor cells in vitro under conditions to permit the cells to obtain a GABAergic neuronal phenotype and to produce GABA.
2. The transplant composition according to statement 1, wherein the GFRalpha agonist is selected from the group consisting of glial cell-derived neurotrophic factor (GDNF), Neurturin (NRTN), Artemin (ARTN) and Persephin (PSPN), Brain-derived neurotrophic factor (BDNF), NGF, GDNF receptor endogenous agonists, BT18, BT13, NT-3, NT-4, CNTF, GFRalphal Agonists, XIB4035, Trk activator, TrkA Agonists, Gamobogic Amine, Amitriptyline, TrkB agonists, N-Acetylserotonin, Amitriptyline, BNN-20BNN-27, Deoxygedunin, 7,8-Dihydroxyflavone, 4′-Dimethylamino-7,8-dihydroxyflavone, Diosmetin, HIOC, LM22A-4, Neurotrophin-3, Neurotrophin-4, Norwogonin, R7 (drug), and 7,8,3′-Trihydroxyflavone.
3. The transplant composition according to statement 2, wherein the GFRalpha agonist is GDNF.
4. The transplant composition according to statement 3, wherein GDNF is present at a concentration of about 1 pM to 100 mM.
5. The transplant composition according to statement 4, wherein GDNF is present at a concentration of about 10 ng/mL.
6. The transplant composition according to any one of the preceding statements, wherein the apoptosis inhibitor is selected from one or more of, a caspase inhibitor selected from the group consisting of Boc-Asp(OMe) fluoromethyl ketone IDN-8066, 7053, 7436 1965 6556 M867 IDN-5370 IDN-7866 pralnacasan z-Vad-FMK, YVAD-FMK, c-DEVD-CHO, Ac-YVAD-CHO, Ac-DVAD-FMK Q-Vd-OPh, CrmA (cowpox virus protein), p35 (Bacoluvirus protein), Z-ATAD-FMK, INF-4E, Z-DQMD-FMK, Az 10417808, Z-LEED-FMK, ZVDK-FMK, z-IETD-FMK, INf-39, Belnacasan, Ac-DEVD-CHO, and Emricasan; or a an inhibitor of a caspase activator selected from the group consisting of Calpain inhibitor 1, Calpeptin, E64, MDL28170, MG101, Acetyl-Calpastatin, and PD 150606.
7. The transplant composition according to statement 6, wherein the apoptosis inhibitor is selected from the group consisting of a caspase inhibitor selected from the group consisting of Boc-Asp(OMe) fluoromethyl ketone IDN-8066, 7053, 7436 1965 6556 M867 IDN-5370 IDN-7866 pralnacasan z-Vad-FMK, YVAD-FMK, c-DEVD-CHO, Ac-YVAD-CHO, Ac-DVAD-FMK Q-Vd-OPh, CrmA (cowpox virus protein), p35 (Bacoluvirus protein), Z-ATAD-FMK, INF-4E, Z-DQMD-FMK, Az 10417808, Z-LEED-FMK, ZVDK-FMK, z-IETD-FMK, INf-39, Belnacasan, Ac-DEVD-CHO, and Emricasan.
8. The transplant composition according to statement 6, wherein the apoptosis inhibitor is a broad-spectrum caspase inhibitor.
9. The transplant composition according to statement 8, wherein the apoptosis inhibitor is Boc-Asp(OMe) fluoromethyl ketone.
10. The transplant composition according to statement 9, wherein Boc-Asp(OMe) fluoromethyl ketone is present at a concentration of about 1 pM to 100 mM.
11. The transplant composition according to statement 10, wherein Boc-Asp(OMe) fluoromethyl ketone is present at a concentration of about 20 μM.
12. The transplant composition according to any one of the preceding statements, wherein the necrosis inhibitor is selected from the group consisting of MS-1, IM-54, GSK-872, 7-Cl-O-Nec1, Necrostatin-1, Necrosulfonamide.
13. The transplant composition according to statement 12, wherein the necrosis inhibitor is selected from the group consisting of MS-1, IM-54, GSK-872, 7-C1-O-Nec1, Necrostatin-1, Necrosulfonamide.
14. The transplant composition according to statement 12, wherein the necrosis inhibitor is a MLKL inhibitor.
15. The transplant composition according to statement 14, wherein the necrosis inhibitor is necrosulfonamide.
16. The transplant composition according to statement 15, wherein necrosulfonamide is present at a concentration of about 1 pM to 100 mM.
17. The transplant composition according to statement 16, wherein necrosulfonamide is present at a concentration of about 50 nM.
18. The transplant composition according to any one of the preceding statements, wherein the pluripotent stem cells, or multipotent stem or progenitor cells are obtained from said mammal.
19. The transplant composition according to any one of the preceding statements, wherein the GABAergic neurons are generated from pluripotent stem cells.
20. The transplant composition according to statement 19, wherein the pluripotent stem cells are iPSCs.
21. The transplant composition according to statement 20, wherein the iPSCs are derived from cells obtained from a biopsy obtained from the mammal.
22. The transplant composition according to statement 21, wherein the cells obtained from a biopsy are dermal fibroblasts.
23. The transplant composition of any one of the preceding statements, wherein the GABAergic neurons are generated by culturing said pluripotent stem cells, or multipotent stem or progenitor cells in the presence of:
   i. at least two SMAD inhibitors from about day 0 to about day 7;
   ii. an activator of sonic hedgehog pathway from about day 0 to about day 21;
   iii. a wnt inhibitor from about day 0 to about day 14;
   iv. a BMP inhibitor from about day 7 to about day 14;
   v. a GABAergic speciation factor from about day 7 to about day 21; and
   vi. a combination of neuronal maturation growth factors comprising BDNF, GDNF and a gamma secretase inhibitor from day 21 to about day 27.
24. The transplant composition according to statement 23, wherein said at least two SMAD inhibitors are selected from the group consisting of Hesperetin, SB431542, SB525334, Galunisertib, GW788388, LY2109761, SB505124, LDN-193189, LDN-193189 HCl, RepSox, A 83-01, DMH1, LDN-212854, ITD 1, LY364947, SD-208, EW-7197, ML347, K02288, A 77-01, SIS3, LDN-214117, R-268712, Pirfenidone, Noggin, Chordin, Gremlin, DAN proteins, and GDF3.
25. The transplant composition according to statement 24, wherein said at least two SMAD inhibitors are LDN193189 and SB431542.
26. The transplant composition according to statement 25, wherein LDN193189 is present at a concentration of about 1 pM to 100 mM and SB431542 is present at a concentration of about 1 pM to 100 mM.
27. The transplant composition according to statement 26, wherein LDN193189 is present at a concentration of about 100 nM and SB431542 is present at a concentration of about 10 μM.
28. The transplant composition according to any one of statements 23-27, wherein said activator of sonic hedgehog signaling is selected from the group consisting of Sonic Hedgehog, GSA10, Purmorphamine, SAG, and SAG dihydrochloride.
29. The transplant composition according to statement 28, wherein said activator of sonic hedgehog signaling is SAG.
30. The transplant composition according to statement 29, wherein SAG is present at a concentration of about 1 pM to 100 mM.
31. The transplant composition according to statement 30, wherein SAG is present at a concentration of about 0.1 μM.
32. The transplant composition according to any one of statements 23-31, wherein said wnt inhibitor is selected from the group consisting of ICG-001, Salinomycin, IWR-1, Wnt-059, ETC-159, iCRT3, IWP2, IWP-4, Pyrvinium Pamoate, iCRT14, FH535, CCT251545, KYA1797K, Wogonin, NCB-0846, Hexachrorophene, PNU-74654, Ky0211, Triptonide, IWP12, Axin, GSK, WAY316606, Shizokaol D, BC2059, PKF115-584, ICG-01, Quercetin, DCA, LY2090314, CHIR99021, SB-216763, NSC668036, QS11, G007-LK, and G244LM.
33. The transplant composition according to statement 32, wherein said wnt inhibitor is IWP2.
34. The transplant composition according to statement 33, wherein IWP2 is present at a concentration of about 1 pM to 100 mM.
35. The transplant composition according to statement 34, wherein IWP2 is present at a concentration of about 5 μM.
36. The transplant composition according to any one of statements 23-35, wherein said BMP inhibitor is selected from the group consisting of Hesperetin, SB431542, SB525334, Galunisertib, GW788388, LY2109761, SB505124, LDN-193189, LDN-193189 HCl, RepSox, A 83-01, DMH1, LDN-212854, ITD 1, LY364947, SD-208, EW-7197, ML347, K02288, A 77-01, SIS3, LDN-214117, R-268712, Pirfenidone, Noggin, Chordin, Gremlin, DAN proteins, and GDF3.
37. The transplant composition according to statement 36, wherein said BMP inhibitor is LDN-193189.
38. The transplant composition according to statement 37, wherein LDN-193189 is present at a concentration of about 1 pM to 100 mM.
39. The transplant composition according to statement 38, wherein LDN-193189 is present at a concentration of about 100 nM.
40. The transplant composition according to any one of statements 23-39, wherein said GABAergic speciation factor is selected from the group consisting of Fibroblasts Growth Factors.
41. The transplant composition according to statement 40, wherein said GABAergic speciation factor is FGF8.
42. The transplant composition according to statement 41, wherein FGF8 is present at a concentration of about 1 pM to 100 mM.
43. The transplant composition according to statement 42, wherein FGF8 is present at a concentration of about 100 ng/mL.
44. The transplant composition according to any one of statements 23-43, wherein said gamma secretase inhibitor is selected from the group consisting of DAPT, RO4929097, Semagecestat, Avagacestat, Dibenzazipine, Ly411575, IMR-1, L-685,458, FLI-06, Crenigacestat, Nirogacestat, MK-0752, Begacestat, BMS299897, Compound W, DBZ, Flurizan, JLK6, MRK560, and PF3084014 hydrobromide.
45. The transplant composition according to statement 44, wherein said gamma secretase inhibitor is DAPT.
46. The transplant composition according to statement 45, wherein DAPT is present at a concentration of about 1 pM to 100 mM.
47. The transplant composition according to statement 46, wherein DAPT is present at a concentration of about 2.5 μM.
48. The transplant composition according to statement 23, wherein said combination of neuronal maturation growth factors comprises BDNF present at a concentration of about 1 pM to 100 mM, GDNF present at a concentration of about 1 pM to 100 mM and DAPT present at a concentration of about 1 pM to 100 mM.
49. The transplant composition according to statement 48, wherein said combination of neuronal maturation growth factors comprises BDNF present at a concentration of about 10 ng/mL, GDNF present at a concentration of about 10 ng/mL and DAPT present at a concentration of about 2.5 μM.
50. The transplant composition according to any one of statements 23-49, wherein said pluripotent stem cells, or multipotent stem or progenitor cells are cultured in the presence of a Rock inhibitor from day 0 for a period of about 24 h.
51. The transplant composition according to any one of statements 1-50, wherein said GABAergic neurons are post-mitotic.
52. The transplant composition according to any one of statements 1-51, wherein said GABAergic neurons express transcripts for Nkx2.1, vGAT, GAD65, GAD67.
53. The transplant composition according to any one of the preceding statements, wherein said GABAergic neurons express GAD65/67, GlyT2 and VGAT.
54. The transplant composition according to any one of the preceding statements, wherein at least 95% of said population of GABAergic neurons express GAD65.
55. The transplant composition according to any one of the preceding statements, wherein at least 95% of said population of GABAergic neurons express VGAT.
56. The transplant composition according to any one of the preceding statements, wherein said GABAergic neurons are capable of secreting GABA in vivo.
57. The transplant composition according to any one of the preceding statements, wherein said GABAergic neurons are capable of functionally integrating with the nervous system of a recipient.
58. The transplant composition according to any one of the preceding statements, further comprising a pharmaceutically acceptable carrier.
59. The transplant composition according to statement 58, wherein said pharmaceutically acceptable carrier is selected from the group consisting of a saline solution or an aqueous buffer.
60. The transplant composition according to any one of the preceding statements, wherein said composition comprises GABAergic neurons at a concentration of about 1000 to 10 million cells/microlitre.
61. The transplant composition according to any one of the preceding statements, wherein said composition comprises GABAergic neurons at a concentration of about 100,000 cells/microlitre.
62. The transplant composition according to any one of the preceding statements wherein the GABAergic neurons are human cells.
63. A method of restoring or reinforcing central inhibition in the nervous system of a mammal comprising administering to the mammal a transplant composition according to any one of statements 1-62.
64. A method of treating a neurological condition, disease or disorder in a mammal comprising administering to the mammal a transplant composition according to any one of statements 1-62.
65. The method according to statement 64, wherein the neurological condition, disease or disorder is characterised by inadequate inhibitory interneuron activity.
66. The method according to statement 64, wherein the neurological condition, disease or disorder is selected from a neurodegenerative disease, neurological injury, or neuropathic pain.
67. The method according to statement 64, wherein said neurological condition, disease or disorder is selected from the group consisting of: Chronic Neuropathic pain, Chronic Inflammatory Pain, Chronic dysfunctional Pain, Epilepsy, Motor neuron disease (ALS, SMA), Parkinson's Disease, Alzheimer's Disease, Stroke, Multiple Sclerosis, Tauopathies (Progressive Supranuclear Palsy, Pick's disease, Cortical Basal Degeneration (CBD), Frontotemporal lobe dementia, FTLD with ALS), Huntington's disease, Alcohol withdrawal and Alcoholism, Diabetes induced brain damage, Head injury, Migraine, Headache, Cluster Headache, Spinal Cord Injury, Ischaemic Damage, Chemotherapy induced pain and chemotherapy induced neuropathy, Schizophrenia, Chronic Depression, Tardive Dyskinesia, Bipolar Disorder, and Neuropathies.
68. A method of treating neuropathic pain in a mammal comprising administering to the mammal a transplant composition according to any one of statements 1-62.
69. The method according to statement 68, wherein said neuropathic pain is associated with sciatica, back pain, cancer pain, diabetic pain, accidental injury, spinal cord injury, peripheral nerve injury.
70. A method of treating allodynia in a mammal, comprising administering to the mammal a transplant composition according to any one of statements 1-62.
71. The method according to any one of statements 63-70, wherein said transplant composition is administered to the central nervous system of the mammal.
72. The method according to any one of statements 63 to 71, wherein said administering comprises injecting said transplant composition into the spinal cord of said mammal.
73. The method according to statement 72, wherein said injecting is stereotactic injection.
74. The method according to any one of statements 63-73, wherein the mammal is a human.
75. A method of delivering GABAergic neurons to a subject in need thereof, said method comprising the steps of:
   a) obtaining a biopsy from said subject and isolating cells from said biopsy;
   b) generating iPSCs from cells isolated in step a);
   c) culturing the iPSCs generated in step b) under conditions to differentiate said iPSCs into GABAergic neurons, wherein said GABAergic neurons express a GABAergic neuronal phenotype and produce GABA;
   d) preparing a transplant composition suitable for injection to a said subject, said transplant composition comprising the GABAergic neurons generated in step c), a GFRalpha agonist, an apoptosis inhibitor, a necrosis inhibitor, and a pharmaceutically acceptable carrier;
   e) administering the transplant composition prepared in step d) to said subject.
76. The method according to statement 75, wherein said subject has inadequate inhibitory interneuron activity or increased excitatory neuron function.
77. The method according to statement 75, wherein said subject has a neurological condition, disease or disorder.
78. The method according to statement 77, wherein the neurological condition, disease or disorder is characterised by inadequate inhibitory interneuron activity.
79. The method according to statement 77, wherein the neurological condition, disease or disorder is selected from a neurodegenerative disease, neurological injury, or neuropathic pain.
80. The method according to statement 77, wherein said neurological condition, disease or disorder is selected from the group consisting of: Chronic Neuropathic pain, Chronic Inflammatory Pain, Chronic dysfunctional Pain, Epilepsy, Motor neuron disease (ALS, SMA), Parkinson's Disease, Alzheimer's Disease, Stroke, Multiple Sclerosis, Tauopathies (Progressive Supranuclear Palsy, Pick's disease, Cortical Basal Degeneration (CBD), Frontotemporal lobe dementia, FTLD with ALS), Huntington's disease, Alcohol withdrawal and Alcoholism, Diabetes induced brain damage, Head injury, Migraine, Headache, Cluster Headache, Spinal Cord Injury, Ischaemic Damage, Chemotherapy induced pain and chemotherapy induced neuropathy, Schizophrenia, Chronic Depression, Tardive Dyskinesia, Bipolar Disorder, and Neuropathies.
81. The method according to statement 75, wherein said subject has neuropathic pain.
82. The method according to statement 81, wherein said neuropathic pain is associated with inflammation, sciatica, back pain, cancer pain, diabetic neuropathy, accidental injury, spinal cord injury, peripheral nerve injury.
83. The method according to statement 75, wherein said subject has allodynia.
84. The method according to any one of statements 75-83, wherein said transplant composition is administered to the central nervous system of the subject.
85. The method according to any one of statements 75 to 84, wherein said administering comprises injecting said transplant composition into the spinal cord of said subject.
86. The method according to statement 85, wherein said injecting is via stereotactic injection.
87. The method according to any one of statements 75-86, wherein the subject is a human.
88. The method according to statements 75-87, wherein the GFRalpha agonist is selected from the group consisting of glial cell-derived neurotrophic factor (GDNF), Neurturin (NRTN), Artemin (ARTN) and Persephin (PSPN), Brain-derived neurotrophic factor (BDNF), NGF, GDNF receptor endogenous agonists, BT18, BT13, NT-3,NT-4, CNTF, GFRalphal Agonists, XIB4035, Trk activator, TrkA Agonists, Gamobogic Amine, Amitriptyline, TrkB agonists, N-Acetylserotonin, Amitriptyline, BNN-20BNN-27, Deoxygedunin, 7,8-Dihydroxyflavone, 4′-Dimethylamino-7,8-dihydroxyflavone, Diosmetin, HIOC, LM22A-4, Neurotrophin-3, Neurotrophin-4, Norwogonin, R7 (drug), and 7,8,3′-Trihydroxyflavone.
89. The method according to statement 88, wherein the GFRalpha agonist is GDNF.
90. The method according to statement 89, wherein GDNF is present at a concentration of about 1 pM to 100 mM.
91. The method according to statement 90, wherein GDNF is present at a concentration of about 10 ng/mL.
92. The method according to any one of statements 75-91, wherein the apoptosis inhibitor is selected from one or more of, a caspase inhibitor selected from the group consisting of Boc-Asp(OMe) fluoromethyl ketone IDN-8066, 7053, 7436 1965 6556 M867 IDN-5370 IDN-7866 pralnacasan z-Vad-FMK, YVAD-FMK, c-DEVD-CHO, Ac-YVAD-CHO, Ac-DVAD-FMK Q-Vd-OPh, CrmA (cowpox virus protein), p35 (Bacoluvirus protein), Z-ATAD-FMK, INF-4E, Z-DQMD-FMK, Az 10417808, Z-LEED-FMK, ZVDK-FMK, z-IETD-FMK, INf-39, Belnacasan, Ac-DEVD-CHO, and Emricasan; or a an inhibitor of a caspase activator selected from the group consisting of Calpain inhibitor 1, Calpeptin, E64, MDL28170, MG101, Acetyl-Calpastatin, and PD 150606.
93. The method according to statement 92, wherein the apoptosis inhibitor is selected from the group consisting of a caspase inhibitor selected from the group consisting of Boc-Asp(OMe) fluoromethyl ketone IDN-8066, 7053, 7436 1965 6556 M867 IDN-5370 IDN-7866 pralnacasan z-Vad-FMK, YVAD-FMK, c-DEVD-CHO, Ac-YVAD-CHO, Ac-DVAD-FMK Q-Vd-OPh, CrmA (cowpox virus protein), p35 (Bacoluvirus protein), Z-ATAD-FMK, INF-4E, Z-DQMD-FMK, Az 10417808, Z-LEED-FMK, ZVDK-FMK, z-IETD-FMK, INf-39, Belnacasan, Ac-DEVD-CHO, and Emricasan.
94. The method according to statement 93, wherein the apoptosis inhibitor is a broad-spectrum caspase inhibitor.
95. The method according to statement 94, wherein the apoptosis inhibitor is Boc-Asp(OMe) fluoromethyl ketone.
96. The method according to statement 95, wherein Boc-Asp(OMe) fluoromethyl ketone is present at a concentration of about 1 pM to 100 mM.
97. The method according to statement 96, wherein Boc-Asp(OMe) fluoromethyl ketone is present at a concentration of about 20 μM.
98. The method according to any one of statements 75-97, wherein the necrosis inhibitor is selected from the group consisting of MS-1, IM-54, GSK-872, 7-C1-O-Nec1, Necrostatin-1, Necrosulfonamide.
99. The method according to statement 98, wherein the necrosis inhibitor is selected from the group consisting of MS-1, IM-54, GSK-872, 7-C1-O-Nec1, Necrostatin-1, Necrosulfonamide.
100. The method according to statement 99, wherein the necrosis inhibitor is a MLKL inhibitor.
101. The method according to statement 100, wherein the necrosis inhibitor is necrosulfonamide.
102. The method according to statement 101, wherein necrosulfonamide is present at a concentration of about 1 pM to 100 mM.
103. The method according to statement 102, wherein necrosulfonamide is present at a concentration of about 50 nM.
104. The method according to statement 21, wherein the cells obtained from said biopsy are dermal fibroblasts.
105. The method of any one of the preceding statements, wherein the GABAergic neurons generated in step c) are generated by culturing said iPSCs, in the presence of:
   i. at least two SMAD inhibitors from about day 0 to about day 7;
   ii. an activator of sonic hedgehog pathway from about day 0 to about day 21;
   iii. a wnt inhibitor from about day 0 to about day 14;
   iv. a BMP inhibitor from about day 7 to about day 14;
   v. a GABAergic speciation factor from about day 7 to about day 21; and
   vi. a combination of neuronal maturation growth factors comprising BDNF, GDNF and a gamma secretase inhibitor from day 21 to about day 27.
106. The method according to statement 105, wherein said at least two SMAD inhibitors are selected from the group consisting of Hesperetin, SB431542, SB525334, Galunisertib, GW788388, LY2109761, SB505124, LDN-193189, LDN-193189 HCl, RepSox, A 83-01, DMH1, LDN-212854, ITD 1, LY364947, SD-208, EW-7197, ML347, K02288, A 77-01, SIS3, LDN-214117, R-268712, Pirfenidone, Noggin, Chordin, Gremlin, DAN proteins, and GDF3.
107. The method according to statement 106, wherein said at least two SMAD inhibitors are LDN193189 and SB431542.
108. The method according to statement 107, wherein LDN193189 is present at a concentration of about 1 pM to 100 mM and SB431542 is present at a concentration of about 1 pM to 100 mM.
109. The method according to statement 108, wherein LDN193189 is present at a concentration of about 100 nM and SB431542 is present at a concentration of about 10 μM.
110. The method according to any one of statements 105-109, wherein said activator of sonic hedgehog signaling is selected from the group consisting of Sonic Hedgehog, GSA10, Purmorphamine, SAG, and SAG dihydrochloride.
111. The method according to statement 110, wherein said activator of sonic hedgehog signaling is SAG.
112. The method according to statement 111, wherein SAG is present at a concentration of about 1 pM to 100 mM.
113. The method according to statement 112, wherein SAG is present at a concentration of about 0.1 μM.
114. The method according to any one of statements 105-113, wherein said wnt inhibitor is selected from the group consisting of ICG-001, Salinomycin, IWR-1, Wnt-059, ETC-159, iCRT3, IWP2, IWP-4, Pyrvinium Pamoate, iCRT14, FH535, CCT251545, KYA1797K, Wogonin, NCB-0846, Hexachrorophene, PNU-74654, Ky0211, Triptonide, IWP12, Axin, GSK, WAY316606, Shizokaol D, BC2059, PKF115-584, ICG-01, Quercetin, DCA, LY2090314, CHIR99021, SB-216763, NSC668036, QS11, G007-LK, and G244LM.
115. The method according to statement 114, wherein said wnt inhibitor is IWP2.
116. The method according to statement 115, wherein IWP2 is present at a concentration of about 1 pM to 100 mM.
117. The method according to statement 116, wherein IWP2 is present at a concentration of about 5 μM.
118. The method according to any one of statements 105-117, wherein said BMP inhibitor is selected from the group consisting of Hesperetin, SB431542, SB525334, Galunisertib, GW788388, LY2109761, SB505124, LDN-193189, LDN-193189 HCl, RepSox, A 83-01, DMH1, LDN-212854, ITD 1, LY364947, SD-208, EW-7197, ML347, K02288, A 77-01, SIS3, LDN-214117, R-268712, Pirfenidone, Noggin, Chordin, Gremlin, DAN proteins, and GDF3.
119. The method according to statement 118, wherein said BMP inhibitor is LDN-193189.
120. The method according to statement 119, wherein LDN-193189 is present at a concentration of about 1 pM to 100 mM.
121. The method according to statement 120, wherein LDN-193189 is present at a concentration of about 100 nM.
122. The method according to any one of statements 105-121, wherein said GABAergic speciation factor is selected from the group consisting of Fibroblasts Growth Factors.
123. The method according to statement 122, wherein said GABAergic speciation factor is FGF8.
124. The method according to statement 124, wherein FGF8 is present at a concentration of about 1 pM to 100 mM.
125. The method according to statement 124, wherein FGF8 is present at a concentration of about 100 ng/mL.
126. The method according to any one of statements 105-125, wherein said gamma secretase inhibitor is selected from the group consisting of DAPT, RO4929097, Semagecestat, Avagacestat, Dibenzazipine, Ly411575, IMR-1, L-685,458, FLI-06, Crenigacestat, Nirogacestat, MK-0752, Begacestat, BMS299897, Compound W, DBZ, Flurizan, JLK6, MRK560, and PF3084014 hydrobromide.
127. The method according to statement 126, wherein said gamma secretase inhibitor is DAPT.
128. The method according to statement 127, wherein DAPT is present at a concentration of about 1 pM to 100 mM.
129. The method according to statement 128, wherein DAPT is present at a concentration of about 2.5 μM.
130. The method according to statement 105, wherein said combination of neuronal maturation growth factors comprises BDNF present at a concentration of about 1 pM to 100 mM, GDNF present at a concentration of about 1 pM to 100 mM and DAPT present at a concentration of about 1 pM to 100 mM.
131. The method according to any one of statements 105-130, wherein said combination of neuronal maturation growth factors comprises BDNF present at a concentration of about 10 ng/mL, GDNF present at a concentration of about 10 ng/mL and DAPT present at a concentration of about 2.5 μM.
132. The method according to any one of statements 105-131, wherein said iPSCs are cultured in the presence of a Rock inhibitor from day 0 for a period of about 24 h.
133. The method according to any one of statements 75-132, wherein said GABAergic neurons are post-mitotic.
134. The method according to any one of statements 75-133, wherein said GABAergic neurons express transcripts for Nkx2.1, vGAT, GAD65, GAD67.
135. The method according to any one of statements 75-134, wherein said GABAergic neurons express GAD65/67, GlyT2 and VGAT.
136. The method according to any one of statements 75-135, wherein at least 95% of said population of GAB Aergic neurons express GAD65.
137. The method according to any one of statements 75-136, wherein at least 95% of said population of GABAergic neurons express VGAT.
138. The method according to any one of statements 75-137, wherein said GABAergic neurons are capable of secreting GABA in vivo.
139. The method according to any one of statements 75-138, wherein said GABAergic neurons are capable of functionally integrating with the nervous system of a recipient.
140. The method according to any one of statements 75-139, wherein said pharmaceutically acceptable carrier is selected from the group consisting of a saline solution or an aqueous buffer.
141. The method according to any one of statements 75-141, wherein said transplant composition comprises GABAergic neurons at a concentration of about 1000 to 10 million cells/microlitre.
142. The method according to statement 141, wherein said composition comprises GABAergic neurons at a concentration of about 100,000 cells/microlitre.
143. A transplant composition according to any one of statements 1-62 for the treatment of inadequate inhibitory interneuron activity or increased excitatory neuron function in a subject.
144. A transplant composition according to any one of statements 1-62 for the treatment of a neurological condition, disease or disorder in a subject.
145. The transplant composition according to any one of statements 1-62 for the use according to statement 144, wherein the neurological condition, disease or disorder is characterised by inadequate inhibitory interneuron activity.
146. The transplant composition according to any one of statements 1-62 for the use according to statement 144, wherein the neurological condition, disease or disorder is selected from a neurodegenerative disease, neurological injury, or neuropathic pain.
147. The transplant composition according to any one of statements 1-62 for the use according to statement 144, wherein said neurological condition, disease or disorder is selected from the group consisting of: Chronic Neuropathic pain, Chronic Inflammatory Pain, Chronic dysfunctional Pain, Epilepsy, Motor neuron disease (ALS, SMA), Parkinson's Disease, Alzheimer's Disease, Stroke, Multiple Sclerosis, Tauopathies (Progressive Supranuclear Palsy, Pick's disease, Cortical Basal Degeneration (CBD), Frontotemporal lobe dementia, FTLD with ALS), Huntington's disease, Alcohol withdrawal and Alcoholism, Diabetes induced brain damage, Head injury, Migraine, Headache, Cluster Headache, Spinal Cord Injury, Ischaemic Damage, Chemotherapy induced pain and chemotherapy induced neuropathy, Schizophrenia, Chronic Depression, Tardive Dyskinesia, Bipolar Disorder, and Neuropathies.
148. A transplant composition according to any one of statements 1-62 for the treatment of neuropathic pain in a subject.
149. The transplant composition according to any one of statements 1-62 for the use according to statement 148, wherein said neuropathic pain is associated with inflammation, sciatica, back pain, cancer pain, diabetic neuropathy, accidental injury, spinal cord injury, peripheral nerve injury.
150. A transplant composition according to any one of statements 1-62 for the treatment of allodynia in a subject.
151. A transplant composition according to any one of statements 1-62 for the use according to any one of statements 143-150, wherein said administering comprises injecting said transplant composition into the spinal cord of said subject.
152. A transplant composition according to any one of statements 1-62 for the use according to statement 151, wherein said injecting is via stereotactic injection.
153. A transplant composition according to any one of statements 1-62 for the use according to any one of statements 143-152, wherein the subject is a human.
154. Use of a transplant composition according to any one of statements 1-62 for the manufacture of a medicament for the treatment of a neurological condition, disease or disorder in a subject.
155. The use according to statement 154, wherein the neurological condition, disease or disorder is characterised by inadequate inhibitory interneuron activity.
156. The use according to statement 154 or 155, wherein the neurological condition, disease or disorder is selected from a neurodegenerative disease, neurological injury, or neuropathic pain.
157. The use according to statement 156, wherein said neurological condition, disease or disorder is selected from the group consisting of: Chronic Neuropathic pain, Chronic Inflammatory Pain, Chronic dysfunctional Pain, Epilepsy, Motor neuron disease (ALS, SMA), Parkinson's Disease, Alzheimer's Disease, Stroke, Multiple Sclerosis, Tauopathies (Progressive Supranuclear Palsy, Pick's disease, Cortical Basal Degeneration (CBD), Frontotemporal lobe dementia, FTLD with ALS), Huntington's disease, Alcohol withdrawal and Alcoholism, Diabetes induced brain damage, Head injury, Migraine, Headache, Cluster Headache, Spinal Cord Injury, Ischaemic Damage, Chemotherapy induced pain and chemotherapy induced neuropathy, Schizophrenia, Chronic Depression, Tardive Dyskinesia, Bipolar Disorder, and Neuropathies.
158. Use of a transplant composition according to any one of statements 1-62 for the manufacture of a medicament for the treatment of neuropathic pain in a subject.
159. The use according to statement 158, wherein said neuropathic pain is associated with inflammation, sciatica, back pain, cancer pain, diabetic neuropathy, accidental injury, spinal cord injury, peripheral nerve injury.
160. Use of a transplant composition according to any one of statements 1-62 for the manufacture of a medicament for the treatment of allodynia in a subject.
161. The use according to any one of statements 154-161, wherein said medicament is formulated for administration to the central nervous system of said subject.
162. The use according to any one of statements 154-161, wherein said medicament is formulated for injection into the spinal cord of said subject.
163. The use according to statement 162, wherein said injection is stereotactic injection.
164. The use according to any one of statements 154-163, wherein the subject is a human.

### BRIEF DESCRIPTION OF DRAWINGS

FIG. 1. Drosophila exhibit thermal allodynia after injury. (A) Uninjured wild-type animals exhibit escape behaviour in response to temperatures>42° C.; this response is dependent on painless and TrpA1, (n=12, 10 animals per replicate). (B) Amputation injury used in this study. (C) Time-course of allodynia response (38° C.) following injury. (D) Dose-response to temperature 14 days after injury, (n=9, 10 animals per replicate). (E) Average Speed of movement for uninjured intact control or animals 7 days after injury in Canton S Data are represented as mean±SEM. ***p<0.001; ns, not significant, two-way ANOVA followed by Tukey's post hoc test for A, C-D, and student's t-test for E.

FIG. 2. TrpA1 is required in ppk+ sensory neurons for allodynia after injury. (A) ppk+ sensory neuron projections in the fly leg. (B) ppk+ cell bodies labelled with Lamin-GFP in the legs. (C) ppk+ sensory neuron projections from the dissected leg to the VNC and brain. (D) Expression of active tetanus toxin (TNT) but not inactive tetanus toxin (iTNT) in ppk+ sensory neurons blocked all adult thermal nocifensive behaviour, (n=9 animals, 10 animals per replicate). (E) TrpA1 and painless mutants are resistant to thermal allodynia (38° C.). (F) TrpA1 is specifically required in ppk+ sensory neurons for allodynia after injury, (n=9, 10 animals per replicate). (G) Re-introduction of TrpA1 specifically in ppk+ sensory neurons rescue allodynia response, (n=9, 10 animals per replicate). Data are represented as mean±SEM. ***p<0.001; ns, not significant, ANOVA followed by Tukey's post hoc test.

FIG. 3. Peripheral injury leads to sensory neuropathy, central sensitisation and augmentation of the nociceptive escape circuit. (A-B) Electrophysiological recordings from DLM, the output of giant fibre system, after (A) stimulation from the intact middle leg, (n≥7), (B) stimulation of the injured leg 7 days after amputation, (n≥7). (C) ppk+ sensory neuropathy is observed after leg amputation. (D) Quantification of sensory neuropathy (ppk1+ projection length) in the amputated leg over time, (n≥7). (E) Adult nociception electrophysiology preparation after injury. (F-H) Leg amputation results in contralateral sensitisation of the escape response circuit with (F, G) increase in escape circuit velocity (velocity difference highlighted in magenta), (F, H) an increase in the duration of the escape response (injured duration highlighted in green) (n≥9). Data are represented as mean±SEM. **p<0.01; ***p<0.001, two-way ANOVA followed by Tukey's post hoc for D, and Mann-Whitney-Wilcoxon tests for G and H.

FIG. 4. GABA gates peripheral activity; peripheral nerve injury reduces GABAergic function. (A) ppk+ sensory neuron projections to the ventral nerve cord (VNC). ppk-Gal4>UAS-CD8-GFP in yellow, anti-GABA in green, nc82 contrast stain in magenta, showing ventral top view close up of anti-GABA and ppk-Gal4>UAS-CD8-GFP colocalisation, and tangential side view close up of anti-GABA and ppk-Gal4>UAS-CD8-GFP colocalisation. (B) Reduction in GABA immunoreactivity after injury of VNC stained for GABA and nc82 from intact uninjured animals and injured animals (7 days after leg amputation), showing ventral top view close up. (C) Imaging of GABAergic interneurons in VNC stained for GABA and nc82 of flies expressing ppk-Gal4>TNT. (D) Imaging of VNC with nuclei-labelled Lamin-GFP (Gad1-Gal4>UAS-Lamin-GFP) and active caspase antibody. (E) Ectopic expression of the caspase inhibitor p35 blocks GABAergic cell death after leg injury. (F-G) GABAergic-specific expression of p35 (Gad1-Gal4>UAS-p35) rescued contralateral sensitisation of the escape response circuit measured by (F) escape circuit velocity, (G) escape response duration, (n≥9). (H) Blocking GABAergic cell death by Gad1-Gal4 driven expression of UAS-p35 prevents neuropathic allodynia behaviour, (n=9, 10 animals per replicate). (I) Nociceptive sensory neuron-specific (ppk-Gal4) knockdown of GABA receptor D-GABA-B-R2, showing it is sufficient to cause thermal allodynia (38° C.) in uninjured flies, (n≥9, 10 animals per replicate). Data are represented as mean±SEM. **p<0.01; ns, not significant, two-way ANOVA followed by Tukey's post hoc test.

FIG. 5: Pure GABAergic neurons can be efficiently derived from hiPSC. (A) Schematic, peripheral nerve injury induces suppression of the pain gate in mice. A potential clinical strategy for the restoration of normal pain is the injection of iPSC-derived GABA neurons in purple. (B) Differentiation protocol for the derivation of Medial Ganglionic Eminence type GABAergic neurons. (C) Brightfield images of GABA neurons during differentiation. DIV28 images of matured GABAergic neurons expressing typical markers, nuclear DAPI, TUBB3, GAD65/67 and merge channels showing purity. (D) FACS analysis of hiPSC-derived GABA neurons at DIV25 of differentiation. Left: Histogram showing TUBB3+ cells in purple (grey, isotype control antibody). Middle: GAD65 expressing cells in purple (grey, isotype control antibody). Right: Dot plot of hiPSC-derived GABA neurons, TUBB3 against GAD65. (E) RNASeq normalised FKPM of GAB Aergic neurons vs hiPSC n=3 hiPSC and n=4 GABAergic neurons (from independent differentiations). (F) Proteomics performed on DIV25 neurons against hiPSC (n=3 per group), Heatmap of normalised iBAQ intensity. (J) Ki67 staining demonstrates some proliferative cells remain at DIV25. (H-I) Imaging of DIV25 neurons. (H) Neurons express VGAT and (I) are GABA immunoreactive.

FIG. 6: Human iPSC derived GABAergic neurons are functional in vitro. (A) GABA concentration in media of DIV25 GABAergic neurons. (B) Normalised FPKM of ionotropic glutamate receptor subunit expression. (C) Representative images of calcium transients following the addition of glutamate and KCl (D) Quantification of calcium transients exhibited by cells (n=50).

FIG. 7: Spinal transplantation of human iPSC derived GABAergic neurons alleviates tactile allodynia. (A) Schematic of strategy of laminectomy based spinal insertion of GABAergic neurons into L1 following spared nerve injury (peripheral neuropathic pain model) and timeline of in vivo experimentation. (B) Normalised von Frey thresholds of injured mice following nerve injury and spinal transplantation of GABAergic neurons. (n=21 vehicle, 29 GABAergic neurons) (C) Stimulus response curves relative to nerve injured mice at 2 months (n=12 vehicle and n=16 GABAergic neurons)(D-E) Open Field Motor analysis of mice following experimentation using open field paradigm and (F) ipsilateral modified Basso Mouse gait analysis scale scores. For these experiments n=9 vehicle, n=13 for GABAergic neuron transplants. (G) Acetone cold allodynia responses. (H) Normalised von Frey thresholds following experiments in naïve mice. (n=11) (I) Normalised von Frey thresholds following nerve injury and injection of iSensory Neurons. n=7. Multiple comparisons performed using repeated measures ANOVA with bonferroni correction for D, E F, G, H, I. Two way ANOVA for B with bonferroni correction. *p<0.05, ** p<0.01, *** p<0.001.

FIG. 8: Human iPSC-derived GABAergic transplants survive and integrate with endogenous circuitry. (A) Schematic and timeline of spinal injection of hiPSC-derived GABAergic neurons following spared nerve injury (peripheral neuropathic pain model). (B-C) Transplanted human GABAergic neurons are identified in the injected ipsilateral dorsal horn which is stained with antibodies to human nuclei (red), anti-IB4 (blue), and CGRP (green) (B-B″) or with antibodies to human nuclei (red) and Synapsin (green) (C-C″). (D-D″) Transplanted human nuclei (red) colocalise with GABA synthesis marker GAD65/67 (green). (E-E″) GABAergic neurons maintain a neuronal phenotype upon transplantation as assessed by TUBB3 and human NCAM colocalisation. (F-F′″) Human cytoplasm (green) is apposed to mouse specific Bassoon (red), which labels mouse pre-synaptic active zones and show presumptive mouse to human graft synapses.

FIG. 8: Human iPSC-derived GABAergic transplants survive and can integrate with endogenous circuitry after 10 weeks. (A) Cells migrate extensively. (B) Cells locate within the dorsal horn. (C) human Nuclei co-localise with NeuN. (D) Human neuronal adhesion molecule co-localises with MAP2. (E) Human Cytoplasm co-localises with TUBB3. (F) GABA is found in Human cytoplasm positive cells. (G) GAD65/67 is found at putative human synapses. (H) Human cell located synaptic vesicles are positive for VGAT. (I) Human Cells are apposed to Mouse Bassoon, RIM2. (J)Voltage gated calcium channels (VGCC) are found in human cells. (K) Liprin is found at putative human synapses. (L-N) Human cytoplasm located synapsin is located apposed to Gephyrin suggesting post synaptic development. (O) SST is found in human cells. (P) Parvalbumin is found in human cells. (Q-R) Ki67 staining in positive control mouse skin and not in human nuclei in spinal cord.

FIG. 10: Injury causes persistent allodynia and ppk+ sensory neuron projections to the ventral nerve cord (VNC) and brain. (A-B) Dose-response of allodynia to temperature (A) 1 day, (B) 7 days after injury, (n=9, 10 animals per replicate). (C) Brain and attached VNC of flies expressing CD8-GFP driven by ppk-Gal4 (ppk-Gal4>UAS-CD8-GFP) (yellow) and co-stained for nc82 (magenta) with (C) ventral top view and (D) ventral top and tangential side view of 2^{nd} lobe of VNC (bottom panels); n=6. (E) Connected brain, attached VNC, and part of femur segment of ppk-Gal4>UAS-CD8-GFP flies (GFP is green), n=5.

FIG. 11: Peripheral injury causes changes in electrophysiological properties of the nociceptive escape circuit. (A-B) The giant fibre response requires higher order brain function. (A) Direct stimulation from giant fibre neuron in flies with the head removed, (n≥7). (B) Stimulation from the intact middle leg in flies with the head removed, (n≥7). (C) Descending stimulation from the head of injured flies still shows decreased response latency, (n≥9). (D) Imaging of central GAB Aergic interneuron loss after peripheral injury; tangential view of VNC stained for GABA (green) and nc82 (magenta) from intact uninjured animals and injured animals (7 days after leg amputation). (E-F) Quantification of anti-GABA foci in intact and injured VNC from ipsilateral and contralateral sides of (E) Canton S, and of flies expressing ppk-Gal4>TNT. Data are represented as mean±SEM. *p<0.05, ** p<0.01 Mann-Whitney-Wilcoxon tests for C, and Student's t-test for E and F.

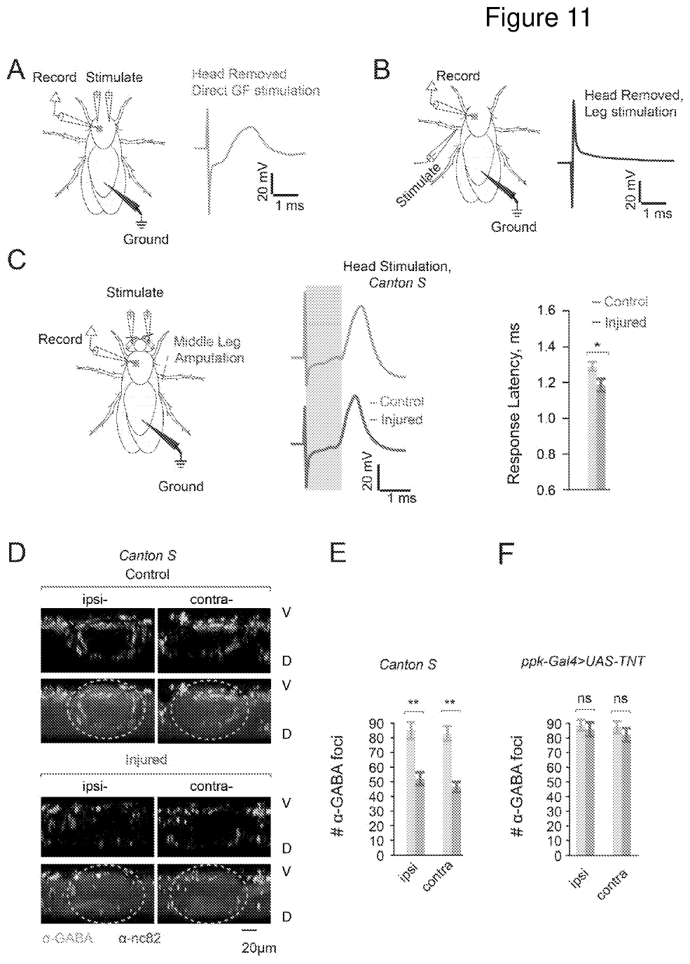

FIG. 12: Peripheral injury reduction in GABA immunoreactivity in the VNC but not the Brain (A-C) Imaging and quantification of GABA foci from VNC and brain stained with anti-GABA (green) and co-stained with anti-nc82 in magenta in control intact and injured flies. (A) Ventral top view of 1^{st} VNC lobe from uninjured and injured animals. (B) 3rd VNC lobe from uninjured and injured animals. (C) Brains from uninjured and injured animals, n≥7 animals per group. (D) Quantification of GABAergic interneurons in VNC 2^{nd} lobe of flies expressing nuclei-labelled Lamin-GFP (Gad1-Gal4>UAS-Lamin-GFP). (E) Quantification of GABA/active caspase double positive cells from control intact and injured VNC of flies expressing nuclei-labelled Lamin-GFP. (F). Quantification of GABA foci in VNC 2^{nd} lobe of flies expressing the caspase inhibitor p35 that blocks GABAergic cell death after leg injury. (G) Knockdown of Rdl but not Grd, GABA-B-R1, or GABA-B-R3 causes allodynia in uninjured flies, (n≥9, 10 flies per replicate). Data are represented as mean±SEM. *p<0.05, ** p<0.01; ns, not significant, student's t-test for A-C, D-F; ANOVA followed by Tukey's post hoc for G.

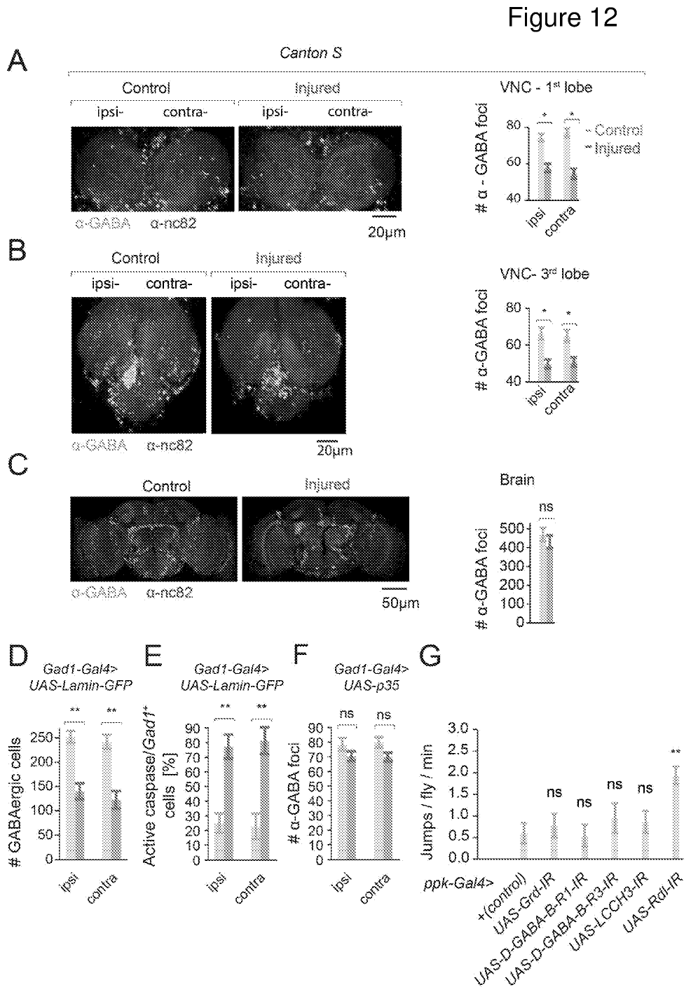

FIG. 13: RNASeq of GABAergic neurons compared to hiPSC and hiPSC derived Sensory neurons. (A) Hierarchical clustering of RNAseq samples yielded expected clusters. (B) RNAseq shows GABAergic neurons express all components required for the synthesis and release of GABA. (C) GABA neurons express multiple ionotropic glutamate receptors including both AMPA and NMDA type. (D) GABAergic neurons are depleted for pluripotency markers and cell cycle and proliferating cell markers. (E) GABAergic neurons are enriched for transcriptional markers of oligodendrocytes but do not express other markers of oligodendrocyte or markers of astrocytes or microglia. (F) Validation by qPCR of select GABA synthesis genes.

FIG. 14: Differentiation signature and subtype classification. (A) Schematic of GABAergic differentiation showing heterogeneity of GABAergic specification. (B) Measurement of classification markers by RNASeq (normalised) with expression expressed semiquantitatively. (C) Neurons were predominantly of a PVALB −ve/SST+ve phenotype at this developmental stage. They sub-classify into multiple phenotypes. Some clearly attained CGE markers and subtype classification is shown for CGE.

FIG. 15: Proteomic analysis of GABAergic neurons relative to hiPSC. (A) Principal component analysis yielded expected sample clustering. (B) Volcano plot of proteomics data. (C) Reactome pathway analysis of differentially expressed genes in GABAergic neurons. (D) GABA synthesis pathway normalised iBAQ values. (E) Western blots of validation of GABAergic markers GAD65 and glycenergic marker GlyT2. N=3 per group.

FIG. 16: Behavioral characterization of transplants. (a) Stimulus response curves at weeks 3 and 4 showing significant analgesia. (b) Data distribution of GABAergic transplants (left), Naïve transplants (middle) and Sensory Transplants (right), (c) Stimulus response curves after 3 weeks of sensory neuron transplant. Sensory neuron transplant but not media caused hyperalgesia. (d) Injection of GABAergic neurons into naïve mice had no effect.

FIG. 17: Efficacy of SST vs PAVB (enriched) hiPSC-GABA neurons to relieve pain in neuropathic mice. (A-B) qPCR analysis of (A) PAVB and (B) SST expression in DIV25 hiPSC-GABA neurons treated or not treated with BMP4 at week 3. (C) hiPSC-GABAergic interneurons treated with BMP4 exhibit a greater analgesic effect when transplanted in nerve injured mice as assessed by von Frey thresholds; (D) Transplanted iGABAergic PAVB enriched neurons show no significant difference compared to their respective baseline control 5 weeks post-transplant (i.e. pain is fully back to normal in SNI animals. Von Frey thresholds are 50% Paw withdrawal thresholds. (Two Way ANOVA compared to media treated unless otherwise stated, Sidak's multiple comparison's test, P<0.05, *, P<0.0001, ****)

### DESCRIPTION OF EMBODIMENTS

### Definitions

As used herein, the term “cell” refers to a single cell as well as to a population of (i.e., more than one) cells. The population may be a pure population comprising one cell type, such as a population of neuronal cells or a population of undifferentiated stem cells. Alternatively, the population may comprise more than one cell type, for example a mixed cell population. It is not meant to limit the number of cells in a population, for example, a mixed population of cells may comprise at least one differentiated cell. In one embodiment a mixed population may comprise at least one differentiated. In the present inventions, there is no limit on the number of cell types that a cell population may comprise.

As used herein, the term “differentiation” as used with respect to cells in a differentiating cell system refers to the process by which cells differentiate from one cell type (e.g., a multipotent, totipotent or pluripotent differentiable cell) to another cell type such as a target differentiated cell). Accordingly, the term “cell differentiation” as used herein, refers to a specialization process or a pathway by which a less specialized cell (e.g. stem cell) develops or matures to possess a more distinct form and function (i.e. more specialized).

As used herein, the term “dedifferentiation” or “dedifferentiated” as used with respect to cells, refers to a process wherein a more specialized cell having a more distinct form and function, and/or limited self-renewal and/or proliferative capacity becomes less specialized and acquires a greater self-renewal and/or proliferative capacity or differentiation capacity (e.g. multipotent, pluripotent etc.). An induced Pluripotent Stem Cell (iPSC) is an example of a de-differentiated cell. Accordingly, dedifferentiation can refer to a process of cellular reprogramming.

As used herein, the term “inducing neuronal differentiation” in reference to a cell refers to changing the default cell type (genotype and/or phenotype) to a non-default cell type (genotype and/or phenotype). Thus “inducing neuronal differentiation in a cell” or “differentiating cells” to permit the cells to obtain a neuronal phenotype includes inducing a cell to have neuronal characteristics, or inducing a cell to divide into progeny cells with neuronal characteristics, that are different from the original identity of the cell, such as genotype (i.e. change in gene expression as determined by genetic analysis such as a PCR or microarray) and/or phenotype (i.e. change in morphology, function and/or expression of a protein, such as β-III tubulin or a plurality of proteins, including a combination of two or more of β-III tubulin, Microtubule Associated Protein 2 (MAP2), synapsin, neurofilament-L, Nestin and N-Cam, Tuj1, GAD65/67, TUJ1, GlyT2 and VGAT).

As used herein, the term “neuron” refers to a differentiated, lineage committed cell of the neural lineage that exhibits the functional and/or phenotypical characteristics of a mature post-mitotic neuron, or a differentiated, lineage committed cell of the neural lineage that requires further maturation, either in vivo or in vitro, in order to exhibit further functional and/or phenotypical characteristics of a mature post-mitotic neuron. Neurons can express one or more of the following markers: tubulin, Microtubule Associated Protein 2 (MAP2), Synapsin, Neurofilament-L, Nestin and N-Cam, Tuj1, GAD65/67, TUJ1, GlyT2 and VGAT. A “GABAergic neuronal phenotype” or “GABAergic neurons” refers to a differentiated, lineage committed cell of the neural lineage that exhibits the functional and/or phenotypical characteristics of a mature post-mitotic neuron and expresses one or more of tubulin, Microtubule Associated Protein 2 (MAP2), Synapsin, Neurofilament-L, Nestin and N-Cam, Tuj1, GAD65/67, TUJ1, GlyT2 and VGAT and produces GABA.

As used herein, the term “inhibit”, “inhibiting” and “inhibition” refers to a reduction, decrease, inactivation, down-regulation, elimination or suppression of an activity or quantity. Accordingly, as used herein, the term “inhibitor” refers to an agent that interferes with (i.e. reduces, decreases, inactivates, down-regulates, eliminates or suppresses) the gene or protein expression of a molecule and/or the activity and/or function of a molecule. For example, in reference to inhibiting a signaling molecule or a signaling molecule's pathway, such as an inhibitor of SMAD signaling, an inhibitor refers to refers to an agent that interferes with the gene or protein expression of an entity involved in the SMAD signaling pathway and/or the activity and/or function of a signaling molecule or the signaling function of the molecule or pathway. Similarly, in reference to an inhibitor of BMP, wnt, gamma secretase, etc., an inhibitor refers to an agent which interferes with the expression or activity or function of BMP, wnt, gamma secretase, etc.

As used herein, the term “contacting” cells with a compound as defined by the present inventions refers to placing the compound in a location that will allow it to touch the cell in order to produce “contacted” cells. The contacting may be accomplished using any suitable method. For example, in one embodiment, contacting is by adding the compound to a container (e.g. tube, vial or culture flask or culture dish etc.) of cells. Contacting may also be accomplished by adding the compound to a culture of the cells.

As used herein, the term “stem cell” refers to a cell that is totipotent or pluripotent or multipotent and is capable of differentiating into one or more different cell types, such as embryonic stems cells, stem cells isolated from organs. As used herein, the term “adult stem cell” refers to a stem cell derived from an organism after birth.

As used herein, the term “neural stem cell” or “NSC” or “neural precursor cell” or “neural progenitor cell” refers to a cell that is capable of becoming neurons, astrocytes, oligodendrocytes, and glial cells in vivo, and neuronal cell progeny and glial progeny in culture.

As used herein, the term “pluripotent” refers to a cell line capable of differentiating into any (or multiple) differentiated cell type (s).

As used herein, the term “multipotent” refers to a cell line capable of differentiating into at least two differentiated cell types.

As used herein, the term “primary cell” is a cell that is directly obtained from a tissue (e.g. blood) or organ of an animal in the absence of culture. Typically, though not necessarily, a primary cell is capable of undergoing ten or fewer passages in vitro before senescence and/or cessation of proliferation.

“Induced pluripotent stem cells (iPSCs) or (iPS cells)” is a designation that pertains to somatic cells that have been reprogrammed or “de-differentiated”, for example, by introducing exogenous genes that confer on the somatic cell a less differentiated phenotype. These cells can then be induced to differentiate into less differentiated progeny. IPS cells have been derived using modifications of an approach originally discovered in 2006 (Yamanaka, S. et al., Cell Stem Cell, 1:39-49 (2007)). For example, in one instance, to create iPS cells, scientists started with skin cells that were then modified by a standard laboratory technique using retroviruses to insert genes into the cellular DNA. In one instance, the inserted genes were Oct4, Sox2, Lif4, and c-myc, known to act together as natural regulators to keep cells in an embryonic stem cell-like state. These cells have been described in the literature. See, for example, Wernig et al., PNAS, 105:5856-5861(2008); Jaenisch et al., Cell, 132:567-582 (2008); Hanna et al., Cell, 133:250-264 (2008); and Brambrink et al., Cell Stem Cell, 2:151-159 (2008). It is also possible that such cells can be created by specific culture conditions (exposure to specific agents) may also be created from a variety of different starting cell types. These references are all incorporated by reference for teaching IPSCs and methods for producing them.

iPS cells have many characteristic features of embryonic stem cells. For example, they have the ability to create chimeras with germ line transmission and tetraploid complementation and they can also form teratomas containing various cell types from the three embryonic germ layers. On the other hand, they may not be identical as some reports demonstrate. See, for example, Chin et al., Cell Stem Cell 5:111-123 (2009) showing that induced pluripotent stem cells and embryonic stem cells can be distinguished by gene expression signatures.

As used herein, the term “cell line,” refers to cells that are cultured in vitro, including primary cell lines, finite cell lines, continuous cell lines, and transformed cell lines, but does not require, that the cells be capable of an infinite number of passages in culture. Cell lines may be generated spontaneously or by transformation.

As used herein, the term “cell culture” refers to any in vitro culture of cells. The term “culturing” refers to the process of growing and/or maintaining and/or manipulating a cell. Included within this term are continuous cell lines (e.g., with an immortal phenotype), primary cell cultures, finite cell lines (e.g., non-transformed cells), and any other cell population maintained in vitro, including oocytes and embryos. As used herein, the terms “primary cell culture,” and “primary culture,” refer to cell cultures that have been directly obtained from cells in vivo, such as from a tissue specimen or biopsy from an animal or human. These cultures may be derived from adults as well as fetal tissue.

As used herein, the terms “culture medium,” and “cell culture medium,” refer to media that are suitable to support the growth of cells in vitro (i.e., cell cultures, cell lines, etc.). It is not intended that the term be limited to any particular culture medium. For example, it is intended that the definition encompass maintenance media as well as other media for the differentiation or specialization of cells. Indeed, it is intended that the term encompass any culture medium suitable for the growth of the cell cultures and cells of interest.

As used herein, the term “kit” refers to any delivery system for delivering materials. In the context of cell differentiation, a kit may refer to a combination of materials for contacting stem cells, such delivery systems include systems that allow for the storage, transport, or delivery of reaction reagents (e.g., compounds, proteins, detection agents (such as probes or antibodies), etc. in the appropriate containers (such as tubes, etc.) and/or supporting materials (e.g., buffers, written instructions for performing cell differentiation, etc.) from one location to another. For example, kits include one or more enclosures (e.g., boxes, or bags, and the like) containing the relevant reaction reagents (such as inhibitors (e.g. SB431542 and LDN193189 wnt inhibitors, bmp inhibitors, apoptosis inhibitors, necrosis inhibitors) and growth factors and cytokines (e.g. GDNF, BDNF)) and/or supporting materials.

As used herein, the term “in vitro” refers to an artificial environment and to processes or reactions that occur within an artificial environment. In vitro environments can consist of, but are not limited to, test tubes and cell cultures. The term “in vivo” refers to the natural environment (e.g., an animal or a cell) and to processes or reaction that occur within a natural environment.

As used herein, the term “marker” or “cell marker” refers to gene or protein that identifies a particular cell or cell type. A marker for a cell may not be limited to one marker; markers may refer to a “pattern” of markers such that a designated group of markers may identify a cell or cell type from another cell or cell type. For example, neurons of the present inventions express one or more markers that distinguish a neuron, e.g. β-III tubulin, Nestin, N-Cam, Tuj1, GAD65/67, TUJ1, GlyT2 and VGAT

The term “derived from” or “established from” or “differentiated from” when made in reference to any cell disclosed herein refers to a cell that was obtained from (e.g., isolated, purified, etc.) a parent cell in a cell line, tissue, or fluids using any manipulation, including single cell isolation, in vivo culture, treatment and/or mutagenesis using for example proteins, chemicals, radiation, infection with virus, transfection with DNA sequences, such as with a morphogen, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure, and the like.

As used herein, the terms “neurodegenerative disorder” and “neurodegenerative disease” are used interchangeably in this document and mean diseases of the nervous system (e.g., the central nervous system or peripheral nervous system) characterized by abnormal cell death. Examples of neurodegenerative conditions include Alzheimer disease, Down's syndrome, frontotemporal dementia, progressive supranuclear palsy, Pick's disease, Niemann-Pick disease, Parkinson's disease, Huntington's disease, dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17)), fragile X (Rett's) syndrome, fragile XE mental retardation, Friedreich's ataxia, myotonic dystrophy, spinocerebellar ataxia type 8, and spinocerebellar ataxia type 12, Alexander disease, Alper's disease, amyotrophic lateral sclerosis (or motor neuron disease), Hereditary spastic paraplegia, mitochondrial disease, ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, ischemic stroke, Krabbe disease, Lewy body dementia, multiple sclerosis, multiple system atrophy, Pelizaeus-Merzbacher disease, Pick's disease, primary lateral sclerosis, Refsum's disease, Sandhoff disease, Schilder's disease, spinal cord injury, spinal muscular atrophy, Steele-Richardson-Olszewski disease, and Tabes dorsalis.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “individual,” “subject,” “host,” and “patient,” are used interchangeably herein and refer to any mammalian subject for whom diagnosis, treatment, or therapy is desired, particularly humans.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above.

Where the terms “comprise”, “comprises”, “comprised” or “comprising” are used in this specification (including the claims) they are to be interpreted as specifying the presence of the stated features, integers, steps or components, but not precluding the presence of one or more other features, integers, steps or components, or group thereof.

A reference herein to a patent document or other matter which is given as prior art is not to be taken as an admission that that document or matter was known or that the information it contains was part of the common general knowledge as at the priority date of any of the claims.

### GABAergic Neuronal Transplant Compositions

Nociception is the sense that allows animals to detect and escape potentially damaging stimuli. In mammals, nociceptive sensory information is integrated and processed in the central nervous system where “pain” is then experienced. This system first evolved over 500 million years ago and the genetic architecture of nociception appears to be under strong selective pressure. The fly larval nocifensive behavior paradigm has been a powerful tool for defining the core conserved genetic architecture of acute nociception. While much work has been done characterizing acute or transient nociceptive sensitisation in the fly larvae, investigating chronic nociceptive states has not yet been possible.

Neuropathic injury leads to a chronic nociceptive sensitization where innocuous stimuli can trigger pain (termed “allodynia”). To identify core underlying mechanisms that could be targeted to treat pain disease, the inventors investigated neuropathic “pain” responses in the fruit fly. Surprisingly, the inventors found that intact animals displayed a robust escape response to temperatures above 42° C., however after injury, animals began exhibiting nocifensive responses to subnoxious temperature (i.e. thermal allodynia). Through a systematic genetic dissection of this response, the inventors have discovered that thermal allodynia was dependent on the conserved TRP channel TrpA1, and loss of central GABAergic inhibition was necessary and sufficient for allodynia. Through investigation the therapeutic potential of restoring spinal inhibition in the context of neuropathic pain, the inventors have surprisingly found that inhibitory GABAergic neuron transplants generated from human induced pluripotent stem cells (hiPSC) when transplanted into neuropathic subjects, not only survived and integrate within the recipient's nervous system (e.g. central nervous system) but, importantly, provided long lasting relief from neuropathic pain without side effects.

The present invention provides a pharmaceutical composition comprising a population of GAB Aergic neurons according to the invention. In another aspect the present invention provides methods for the generation of GABAergic neurons. In a preferred embodiment, the present invention provides a pharmaceutical composition comprising a population of GABAergic neurons produced according to the methods described herein.

The pharmaceutical composition may generally include one or more pharmaceutically acceptable and/or approved carriers, additives, antibiotics, preservatives, adjuvants, diluents and/or stabilizers. Such auxiliary substances can be water, saline, glycerol, ethanol, wetting or emulsifying agents, pH buffering substances, or the like. Suitable carriers are typically large, slowly metabolized molecules such as proteins, polysaccharides, polylactic acids, polyglycollic acids, polymeric amino acids, amino acid copolymers, lipid aggregates, or the like. This pharmaceutical composition can contain additional additives such as mannitol, dextran, sugar, glycine, lactose or polyvinylpyrrolidone or other additives such as antioxidants or inert gas, stabilizers or recombinant proteins (e. g. human serum albumin) suitable for in vivo administration.

As used herein, the term “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In one aspect, the present invention provides a pharmaceutical composition comprising a population of GABAergic neurons according to the invention for use in the treatment of pain in a subject. In a preferred embodiment, the pain is neuropathic pain. The invention also relates to a method for treating pain comprising the step of administering a therapeutically effective amount of a pharmaceutical composition comprising a population of GABAergic neurons according to the invention. In a preferred embodiment, the pain is neuropathic pain.

Another aspect of the invention relates to a population of GABAergic neurons of the invention as described herein, for use in treating a neurodegenerative disease or an injury to the central or peripheral nervous system. The invention also relates to a method for treating a neurodegenerative disease or an injury to the central or peripheral nervous system comprising the step of administering a therapeutically effective amount a population of neurons as described above.

In the context of the invention, the term “treating” or “treatment”, as used herein, refers to a method that is aimed at delaying or preventing the onset of a pathology, at reversing, alleviating, inhibiting, slowing down or stopping the progression, aggravation or deterioration of the symptoms of the pathology, at bringing about ameliorations of the symptoms of the pathology, and/or at curing the pathology.

As used herein, the term “therapeutically effective amount” refers to any amount of a transplant composition or number GABAergic neurons prepared according to the methods described herein (or a population thereof or a pharmaceutical composition thereof) that is sufficient to achieve the intended purpose. Effective dosages and administration regimens can be readily determined by good medical practice based on the nature of the pathology of the subject, and will depend on a number of factors including, but not limited to, the extent of the symptoms of the pathology and extent of damage or degeneration of the tissue or organ of interest, and characteristics of the subject (e.g., age, body weight, gender, general health, and the like).

In one embodiment the present invention is directed towards a transplant composition comprising a population of GABAergic neurons, a GFRalpha agonist, and at least one cell death inhibitor, wherein said GABAergic neurons are generated by differentiating pluripotent stem cells, or multipotent stem or progenitor cells in vitro under conditions to permit the cells to obtain a GABAergic neuronal phenotype and to produce GABA.

In one embodiment the present invention is directed towards a transplant composition comprising a population of GAB Aergic neurons, a GFRalpha agonist, an apoptosis inhibitor, and a necrosis inhibitor, wherein said GABAergic neurons are generated by differentiating pluripotent stem cells, or multipotent stem or progenitor cells in vitro under conditions to permit the cells to obtain a GABAergic neuronal phenotype and to produce GABA.

For therapy, iPSC-derived GABAergic neurons produced according to methods described and exemplified herein and pharmaceutical compositions according to the invention may be administered via any appropriate route. The dose and the number of administrations can be optimized by those skilled in the art in a known manner.

For example, dosage amounts can vary from about 100; 500; 1,000; 2,500; 5,000; 10, 000; 20,000; 50,000; 100,000; 500,000; 1,000,000; 5,000,000 to 10,000,000 cells or more (or any integral value therebetween); with a frequency of administration of, e.g., once per day, twice per week, once per week, twice per month, once per month, once per year, twice per year, once every two, three, four or five months, depending upon, e.g., body weight, route of administration, severity of disease, etc. In one embodiment, the preferred dose is 100,000 cells/microlitre. In another embodiment, the compositions of the present invention comprises 2.5 million cells.

The cells described herein can be suspended in a physiologically compatible carrier for transplantation. As used herein, the term “physiologically compatible carrier” refers to a carrier that is compatible with the other ingredients of the formulation and not deleterious to the recipient thereof. Those of skill in the art are familiar with physiologically compatible carriers. Examples of suitable carriers include cell culture medium (e.g., Eagle's minimal essential medium), phosphate buffered saline, Hank's balanced salt solution+/−glucose (HBSS), and multiple electrolyte solutions such as Plasma-Lyte™ A (Baxter).

In one embodiment, the GFRalpha agonist in the transplant composition is selected from any one or more of the group consisting of: GDNF, Brain-derived neurotrophic factor (BDNF), NGF, Neurturin, Artemin, Persephin, GDNF receptor endogenous agonists, BT18, BT13, NT-3, NT-4, CNTF, GFRalphal Agonists, XIB4035, Trk activator, TrkA Agonists, Gamobogic Amine, Amitriptyline, TrkB agonists, N-Acetylserotonin, Amitriptyline, BNN-20BNN-27, Deoxygedunin, 7,8-Dihydroxyflavone, 4′-Dimethylamino-7,8-dihydroxyflavone, Diosmetin, HIOC, LM22A-4, Neurotrophin-3, Neurotrophin-4, Norwogonin, R7 (drug), and 7,8,3′-Trihydroxyflavone. In one embodiment, the GFRalpha agonist is selected from the group consisting of glial cell-derived neurotrophic factor (GDNF), Neurturin (NRTN), Artemin (ARTN) and Persephin (PSPN). In a preferred embodiment, the GFRalpha agonist is GDNF.

In one embodiment, the apoptosis inhibitor in the transplant composition is selected from one or more of, a caspase inhibitor selected from the group consisting of Boc-Asp(OMe) fluoromethyl ketone IDN-8066, 7053, 7436 1965 6556 M867 IDN-5370 IDN-7866 pralnacasan z-Vad-FMK, YVAD-FMK, c-DEVD-CHO, Ac-YVAD-CHO, Ac-DVAD-FMK Q-Vd-OPh, CrmA (cowpox virus protein), p35 (Bacoluvirus protein), Z-ATAD-FMK, INF-4E, Z-DQMD-FMK, Az 10417808, Z-LEED-FMK, ZVDK-FMK, z-IETD-FMK, INf-39, Belnacasan, Ac-DEVD-CHO, and Emricasan; or a an inhibitor of a caspase activator selected from the group consisting of Calpain inhibitor 1, Calpeptin, E64, MDL28170, MG101, Acetyl-Calpastatin, PD 150606. In a preferred embodiment the apoptosis inhibitor is Boc-Asp(OMe) fluoromethyl ketone.

In another embodiment, the direct inhibition of Caspase genes and pathways may be achieved by genetic engineering of the GABAergic neurons prepared according to the methods described herein or the cells from which they are derived such as by—TALENS, CRISPR-Cas9, or RNAi to promote cellular survival.

In one embodiment, the necrosis inhibitor in the transplant composition is selected from one or more of MS-1, IM-54, GSK-872, 7-Cl-O-Nec1, Necrostatin-1, Necrosulfonamide. In a preferred embodiment, the necrosis inhibitor is Necrosulfonamide.

In another embodiment, the direct inhibition of necroptosis genes and pathways and pathways may be achieved by genetic engineering of the GABAergic neurons prepared according to the methods described herein or the cells from which they are derived such as by—TALENS, CRISPR-Cas9, or RNAi to promote cellular survival.

In another embodiment, the transplant composition of the present invention comprises or may be administered with one or more inhibitors of excitotoxic induced apoptosis or necroptosis selected from the group consisting of Amantadine, Memantine, Ketamine hydrochloride, pethidine, tramadol, methadone, dectropoxyphene, nitrous oxide, dextromethorphan, AP5, AP7, CPPene, Selfotel, Ethanol, Minocycline, Atomoxetine, AZD6765, Agmatine, Chlorophorm, Dextrallorphan, Dextrorphan, Diphenidine, Dizocilpine, Eticyclidine, GAcyclidine, Ketamine (other forms), Magnesium, Methoxetimine, Nitormemantine, PD-137899, Phencyclidine, Rolicyclidine, Tenocyclidine, Tiletamine, Neramexane, Elipradol, Etoxadrol, Dexoxadrol, WMS-2539, NEFA, delucemine, 8A-PDHQ, Aptiganel, HU-211, Huperzine A, Ibogaine, Remacemide, Rhynchophylline, GABApentin, Rapastinel, NRX-1074, 7-chlorkynurenic acid, 4-Chlorkyurenine, 5,7-Dichlorokynurenic acid, Kynurenic acid, TK-40, 1-Aminocyclopropanearboxylic acid, L-Phenylalanine, and Xenon; one or more inhibitors of excitability selected from Bupivacaine, Lidocaine, Cocaine, Lamotrigine, Paraldehyde, Stiripentol, Phenobarbitol, Primidone, Methylphenobarbitol, pentobarbital, Benzodiazepines (Clobazam, Clonazepam, Clozrazepate, Diazepam, Midazolam, Lorazepam, Nitrazepam, Temazepam, Nimetazepam)Potassium Bromide, Felbamate, Carboxamides (Carbamazepine, Oxcarbazepine, Esclicarbazepine Acetate, Valproates (Valporic Acid, Sodium Valporate, Divaproex sodium, Vigabatrin, Progabide, Tiagabine, Topiramate, Pregabalin, Ethotoin, Phenytoin, Mephenytoin, Fosphenytoin, Paramethadone, Trimethadione, Ethadione, Becalamide, Primidone, Brivaracetam, Etiracetam, Levetircetem, Slectracetem, Ethosuximide, Phensuximide, Mesuximide, Acetazolamide, Sultiame, Methazolamide, Zonisamide, Pheneturide, Phenacemide, Valpromide, Valnoctamide, Perampanel, Stiripentol, Pyroxidine, Isoflurane, Levoflurane, CNV1014802, Funapide, Prilocaine, lontocaine, Levobupivacaine, Butanilicaine, Carticaine, Dibucaine, Etidocaine, Mepivacaine, Prilocaine, Trimecaine, Amylocaine, Cyclomethylcaine, alpha-Eucaine, Beta-Eucaine, Hexylcaine, Isobucaine, Piperocaine, Orhtocaine, Benzocaine, Butamibe, Chloroprocaine, Lucaine, Dimethocaine, Meprylcaine, Lucaine, Nitrocaine, Orthocaine, Propoxycaine, Novocaine, Proxymetacaine, Risocaine, Tetracaine. Raxatrigine, Tricyclic antidepressants (amitriptyline, Nortriptyline, DSP-2230, Mexilitine, Flupirtine, ziconotide; or any drug inhibiting peripheral activity including Opium and Opioids, Non-steroidal anti-inflammatories, Paracetamol, Acetenalidide, Capsaicin, Menthol, Cannabis and Cannibinoids.

The amount of each of the aforementioned agonists and inhibitor required to be supplied may be readily determined by the person skilled in the art. For example, the level of inhibition of caspase or caspase signaling and/or the extent apoptosis or necrosis may be determined by routine assays. The concentrations of the GFRalpha agonist, apoptosis inhibitor and necrosis inhibitor to be used in the transplant compositions and methods (including but not limited to cell culture media and kits) may be readily ascertained having regard to neuronal cell viability and function, both of which may be readily assessed using assays known to the skilled addressee together with the assays described herein, and adjusted accordingly.

In embodiments of the invention, the aforementioned inhibitors and agonists may be present in concentrations ranging from picomolar to micromolar concentrations.

In another embodiment, the GABAergic neurons of the transplant compositions of the present invention express one or more of β-III tubulin, Microtubule Associated Protein 2 (MAP2), Synapsin, Neurofilament-L, Nestin and N-Cam, Tuj1, GAD65/67, TUJ1, GlyT2 and VGAT. In a preferred embodiment, the GABAergic neurons express VGAT. In another embodiment, the GABAergic neurons express Glyt2 and GAD65 and GAD67. In another embodiment, the GABAergic neurons express VGAT, Glyt2 and GAD65 and GAD67.

In another embodiment, the GABAergic neurons produce GABA in vivo. In another embodiment, the GABAergic neurons secrete GABA at concentrations described in the examples set forth herein.

In another aspect the present invention provides dosage forms of the transplant compositions of the present invention. In some embodiments, these dosage forms comprise ready-to-administer compositions. The term “ready-to-administer” as used herein means that the drug solution is sterile and suitable for direct intravenous infusion or injection and no intermediate steps of dilution or reconstitution are required before parenteral administration of the drug solution to the patient. The aqueous drug solution can be directly administered parenterally from the container of the dosage form. The term “ready-to-administer” is synonymous with “ready-to-infuse” or ready-to-inject”.

Methods of Preparing GABAergic Neurons

The present invention also relates to methods for the generation of GABAergic neurons and the following aspects and embodiments may be used in conjunction, either individually or in any suitable combination.

Because of the potential of differentiated cells derived from stem cells in countless therapeutic applications, directing or promoting the differentiation of stem cells in culture toward a specific somatic cell fate is of great interest. Human stem cells offer great promise for cell-replacement therapies and cell screening for therapeutics. Recent advances in somatic cell reprogramming to induced pluripotent stem cells (iPSCs) has opened the door to generating patient-specific cells for regenerative medicine and disease modelling.

It has now been surprisingly found that contacting cells which are undergoing differentiation or maturation towards a GABAergic neuronal phenotype with agents which activate BMP signalling, promotes differentiation of progenitor cells towards a GABAergic neuronal phenotype wherein such cells have decreased expression of somatostatin and increased expression of parvalbumin. In other words, these factors which induce BMP signalling induce or direct the differentiation of stem or progenitor cells towards GABAergic neurons with a more favourable inhibitory phenotype. Thus, the use of factors which augment BMP signalling in a culture system for directing the differentiation of stem or progenitor cells towards an inhibitory GABAergic neuronal phenotype, has thus been envisaged.

Therefore, according to another aspect of the invention, there is provided a method for producing GABAergic neurons which have increased expression of parvalbumin, the method comprising culturing a population of cells undergoing neuronal differentiation at, and for a time and under conditions sufficient for inducing BMP signalling in said population of cells sufficient to differentiate the population of cells into cells having a GABAergic neuronal phenotype with elevated expression of parvalbumin compared to cells cultured under conditions wherein BMP signalling is not induced.

While a number of agents which induce BMP signalling within cells are known, according to a preferred embodiment, the population of differentiating cells is cultured for a time and under conditions sufficient for inducing BMP signalling in said population of cells sufficient to differentiate the population of cells into cells having a GABAergic neuronal phenotype and elevated expression of parvalbumin, by contacting the population of cells with one or more agents which induce BMP signalling. Preferably, the one or more agents are selected from canonical ligands for the BMP Type I and/or Type II receptor(s) and activates BMP signalling via binding to either or both receptors. In another embodiment, the one or more agents binds to a BMP Type I receptor (BMPR-I) and BMP Type I receptor (BMPR-II) and/or activates SMAD1/5/8 signalling pathway.

In one embodiment, the one or more agents which induce BMP signalling comprises BMP4. In a preferred embodiment the agent which induces BMP signalling is BMP4. In another embodiment the one or more agents which induce BMP signalling is/are selected from the group consisting of: ventromorphins selected from the group consisting of SJ000291942, SJ000063181 and SJ000370178, isoliquiritigenin (SJ000286237), a BMP sensitizer (PD407824), BMP2, BMP5, BMP4 BMP6, BMP7, BMP9, BMP10, BMP15, BMP co-activators FK506 (Tacrolimus) and CK2.3 (CK2 inhibitor), engineered BMPs, inhibitors of endogenous BMP antagonists Noggin and Chordin (including neutralizing antibodies such as Anti-Noggin, Anti-Chordin antibodies), Fasudil Hydrochloride, lovastatin, Simvastatin (which can enhance BMP expression), pentoxifyline, sildenafil, rolipram (which enhance the effects of BMP through phosphodiesterase inhibition).

In another embodiment the one or more agents which activates induces BMP signalling and/or SMAD1/5/8 signalling inhibits SMURF1 (the endogenous inhibitor of SMURF1).

The concentrations of agent(s) which may be employed to effect induction of BMP signalling in a population of retinal progenitor cells sufficient to differentiate the population of cells into cells undergoing neuronal differentiation, may be determined by the skilled addressee according to the methods described herein. In one embodiment, the population of cells are cultured in a cell culture medium comprising one or more agents which induce BMP signalling present at a concentration of about 1 pM to about 100 μM. In another embodiment, the one or more agents which induce BMP signalling are present at a concentration of about 5 ng/mL to about 50 ng/mL. More preferably, the one or more agents which induce BMP signalling are present at a concentration of about 10 ng/mL. In a preferred embodiment, BMP4 is present at an amount of 10 ng/mL

In the process of preparing GABAergic neurons from a population of iPSCs, as explained in more detail hereinbelow, the inventors have surprisingly found that exposure of cells undergoing neuronal differentiation, or maturation, towards a GABAergic neuronal phenotype with an agent which activates BMP signalling during a period from about day 14 to about day 21 of culture, demonstrated a 6-fold increase of parvalbumin expression compared to cells which were either not treated or treated during from about day 14 to about day 28 or from about day 21 to about day 28, and to a 50% reduction of somatostatin.

It has further been surprisingly found that when cells which are undergoing differentiation or maturation towards a GABAergic neuronal phenotype are contacted with one or more agents which activate BMP signalling the during a period from about day 14 to about day 21 of culture, such cells display a significantly more potent long-term analgesic response when employed therapeutically for the treatment of pain.

Accordingly, in another embodiment, cells which are undergoing differentiation or maturation towards a GABAergic neuronal phenotype are contacted with one or more agents which activate BMP signalling the during a period from about day 14 to about day 21 of culture. In another embodiment, the cells which are undergoing differentiation or maturation towards a GABAergic neuronal phenotype are contacted with one or more agents which activate BMP signalling for the duration of a period from about day 14 to about day 21 of culture. In a preferred embodiment, the one or more agents comprises BMP. In another embodiment, the cells which are undergoing differentiation or maturation towards a GABAergic neuronal phenotype are contacted with BMP4 from about day 14 to about day 21 of culture.

In one embodiment, the GAB Aergic neurons of the present invention are prepared according to the following procedure:

hiPSC are dissociated with TripLE (Invitrogen) and grown for 2 weeks in suspension into ultra-low attachment binding plates to allow the formation of embryoid bodies (EBs). For neural induction, cells are treated with SMAD inhibitors LDN193189 (100 nM, day 0 to day 14) and SB431542 (10 μM, day 0 to day 10). For MGE (Medial Glanglionic eminence) induction, cells were treated with Wnt inhibitor IWP2 (5 μM, day 0 to day 7), SHH activator SAG (Smoothened Agonist—0.1 μM, day 0 to day 21) and growth factor FGF8 (100 ng/mL, day 8 to day 21). Rock inhibitor is added on the first day of differentiation. After 2 weeks in suspension, EBs are transferred to polyornithine (PLO) and fibronectin (FN) coated surfaces. At day 21, EBs were dissociated and cells were replated on PLO/FN coated plates on differentiation media containing 10 ng/mL BDNF, 10 ng/mL GDNF and 2.5 μM gamma secretase inhibitor DAPT for further differentiation and maturation.

According to the forgoing procedure, hiPSCs may be induced to differentiate in ultra-low attachment 24 well plates (each well=1.9 cm2). Optimal cell density is about 600,000 cells per well. Embryoid bodies are plated in Fibronectin-coated 12 well plates at d14 (3.8 cm2). Optimal number of EB's per well is about 10. Cells are then dissociated and replated at d21 (12 well plates). Optimal cell density is about 30,000 cells per well. One of skill in the art will readily appreciate that the optimal densities of cells, or EB's, may be applied to culture vessels of different formats and sizes (e.g. tissue culture flasks, petri dishes etc.).

Confirmation of production of GABA may be assessed using methods described and exemplified herein including analysis of transcripts related to GABA and secretion of GABA into cell culture medium.

Methods of Treatment

Another aspect of the invention relates to transplant compositions of the present invention comprising a population of GABAergic neurons as described herein, for use in treating a neurological condition, disease or disorder in a mammal. The invention also relates to a method for treating a neurological condition, disease or disorder in a mammal comprising the step of administering a therapeutically effective amount a transplant composition as described herein to a subject in need thereof. The invention also relates to use of a transplant composition as described herein, or a population of GABAergic neurons prepared according to methods described herein for the manufacture of a medicament for treating a neurological condition, disease or disorder in a mammal.

In the context of the invention, the term “treating” or “treatment”, as used herein, refers to a method that is aimed at delaying or preventing the onset of a pathology, at reversing, alleviating, inhibiting, slowing down or stopping the progression, aggravation or deterioration of the symptoms of the pathology, at bringing about ameliorations of the symptoms of the pathology, and/or at curing the pathology.

As used herein, the term “therapeutically effective amount” refers to any amount of the transplant composition according to the invention that is sufficient to achieve the intended purpose. Effective dosages and administration regimens can be readily determined by good medical practice based on the nature of the pathology of the subject, and will depend on a number of factors including, but not limited to, the extent of the symptoms of the pathology and extent of damage or degeneration of the tissue or organ of interest, and characteristics of the subject (e.g., age, body weight, gender, general health, and the like).

For therapy, the transplant compositions according to the invention may be administered via any appropriate route. The dose and the number of administrations can be optimized by those skilled in the art in a known manner.

In one embodiment the present invention provides a method of treating neurological condition, disease or disorder in a mammal. In another embodiment the neurological disease disorder or condition is associated with excitotoxicity requiring restoration or reinforcement of inhibition. In one embodiment, the neurological condition, disease or disorder is selected from the group consisting of: Neuropathic pain (including Chronic Neuropathic Pain), Chronic Inflammatory Pain, Chronic dysfunctional Pain, Epilepsy, Motor neuron disease (ALS, SMA), Parkinson's Disease, Alzheimer's Disease, Stroke, Multiple Sclerosis, Tauopathies (Progressive Supranuclear Palsy, Pick's disease, CBD, FTLD, FTLD with ALS), Huntington's disease, Alcohol withdrawal and Alcoholism, Diabetes induced brain damage, Head injury, Migraine, Headache, Cluster Headache, Spinal Cord Injury, Ischaemic Damage, Chemo induced pain and chemo induced neuropathy, Schizophrenia, Chronic Depression, Tardive Dyskinesia, Bipolar Disorder, and Neuropathies.

In one embodiment, the GABAergic neurons as described herein or the transplant composition or population of GABAergic neurons as described herein is administered to the central nervous system of a subject. In one embodiment, the transplant composition or population of GABAergic neurons is administered to the spinal cord of a subject. In another embodiment, the transplant composition or population of GABAergic neurons is administered to the brain of a subject. In another embodiment the transplant composition or population of GABAergic neurons is administered to a dorsal root ganglion of a subject.

Where the invention relates to transplant compositions of the present invention comprising a population of GABAergic neurons as described herein, or a population of GABAergic neurons as described herein for use in treating a neurological condition, disease or disorder in a mammal or uses for the manufacture of a medicament for the treatment of a neurological condition, disease or disorder in a mammal, the composition or medicament may be formulated for administration to the central nervous system of a subject. In one embodiment, the transplant composition or medicament is formulated for administration to the spinal cord of a subject. In another embodiment, the transplant composition or medicament is formulated for administration to the brain of a subject. In another embodiment the transplant composition or medicament is formulated for administration to a dorsal root ganglion of a subject.

In one embodiment, the present invention provides a method of treating pain in a subject in need thereof comprising administering a therapeutically effective amount of a population GABAergic neurons prepared according to the methods described herein or a transplant composition as described herein to said subject. In one embodiment, the present invention provides a population of GABAergic neurons prepared according to methods described herein or a transplant composition as described herein for the for the treatment of pain in a subject. In one embodiment, the present invention provides use of a population of GABAergic neurons prepared according to methods described herein or a transplant composition as described herein for the manufacture of a medicament for the treatment of pain in a subject. In a preferred embodiment the pain is neuropathic pain.

In one embodiment, the present invention provides a method of treating a disease or disorder associated with neuronal excitability in a subject in need thereof comprising administering a therapeutically effective amount of a population GABAergic neurons prepared according to the methods described herein or a transplant composition as described herein to said subject. In one embodiment, the present invention provides a population of GABAergic neurons prepared according to methods described herein or a transplant composition as described herein for the treatment of a disease or disorder associated with neuronal excitability in a subject in need thereof. In one embodiment, the present invention provides use of a population of GABAergic neurons prepared according to methods described herein or a transplant composition as described herein for the manufacture of a medicament for the treatment of a disease or disorder associated with neuronal excitability in a subject in need thereof.

Kits

The present invention provides a kit for the preparation of a transplant compositions described herein. In one embodiment, the kit provides at least one GFRalpha agonist, and at least one cell death inhibitor, together with a population of GAB Aergic neurons. In one embodiment, the kit provides at least one GFRalpha agonist, at least one apoptosis inhibitor, and at least one necrosis inhibitor, together with a population of GABAergic neurons.

In another embodiment the kit comprises pluripotent stem cells, or multipotent stem or progenitor cells, in place of the population of GABAergic neurons, together with cell culture reagents as described herein for differentiating the cells to obtain a GABAergic neuronal phenotype and to produce GABA. In another embodiment, the kit further comprises one or more agents which activate BMP signaling. In one embodiment, the one or more agents which induce BMP signalling comprises BMP4. In a preferred embodiment the agent which induces BMP signalling is BMP4. In another embodiment the one or more agents which induce BMP signalling is/are selected from the group consisting of: ventromorphins selected from the group consisting of SJ000291942, SJ000063181 and SJ000370178, isoliquiritigenin (SJ000286237), a BMP sensitizer (PD407824), BMP2, BMPS, BMP4 BMP6, BMP7, BMP9, BMP10, BMP15, BMP co-activators FK506 (Tacrolimus) and CK2.3 (CK2 inhibitor), engineered BMPs, inhibitors of endogenous BMP antagonists Noggin and Chordin (including neutralizing antibodies such as Anti-Noggin, Anti-Chordin antibodies), Fasudil Hydrochloride, lovastatin, Simvastatin (which can enhance BMP expression), pentoxifyline, sildenafil, rolipram (which enhance the effects of BMP through phosphodiesterase inhibition).

In another embodiment the one or more agents which activates induces BMP signalling and/or SMAD1/5/8 signalling inhibits SMURF1 (the endogenous inhibitor of SMURF1).

In another embodiment the kit further comprises instructions for the differentiation of GABAergic neurons from pluripotent stem cells, or multipotent stem or progenitor cells according to the methods described herein and instructions for the preparation of the transplant composition comprising such GABAergic neurons.

In addition to inhibitors and agonists, the kits of the present invention may further comprise one or more of the following: a culture medium, at least one cell culture medium supplement, an agent for inhibiting or increasing expression of one or more gene products, and at least one agent for detecting expression of a marker of neuronal differentiation.

In preferred embodiments, the GFRalpha agonist in the kit is selected from any one of the group consisting of: GDNF, Brain-derived neurotrophic factor (BDNF), NGF, Neurturin, Artemin, Persephin, GDNF receptor endogenous agonists, BT18, BT13, NT-3, NT-4, CNTF, GFRalphal Agonists, XIB4035, Trk activator, TrkA Agonists, Gamobogic Amine, Amitriptyline, TrkB agonists, N-Acetylserotonin, Amitriptyline, BNN-20BNN-27, Deoxygedunin, 7,8-Dihydroxyflavone, 4′-Dimethylamino-7,8-dihydroxyflavone, Diosmetin, HIOC, LM22A-4, Neurotrophin-3, Neurotrophin-4, Norwogonin, R7 (drug), and 7,8,3′-Trihydroxyflavone. In one embodiment, the GFRalpha agonist is selected from the group consisting of glial cell-derived neurotrophic factor (GDNF), Neurturin (NRTN), Artemin (ARTN) and Persephin (PSPN). In a preferred embodiment, the GFRalpha agonist is GDNF.

In one embodiment, the apoptosis inhibitor in the transplant composition is selected from one or more of, a caspase inhibitor selected from the group consisting of Boc-Asp(OMe) fluoromethyl ketone IDN-8066, 7053, 7436 1965 6556 M867 IDN-5370 IDN-7866 pralnacasan z-Vad-FMK, YVAD-FMK, c-DEVD-CHO, Ac-YVAD-CHO, Ac-DVAD-FMK Q-Vd-OPh, CrmA (cowpox virus protein), p35 (Bacoluvirus protein), Z-ATAD-FMK, INF-4E, Z-DQMD-FMK, Az 10417808, Z-LEED-FMK, ZVDK-FMK, z-IETD-FMK, INf-39, Belnacasan, Ac-DEVD-CHO, and Emricasan; or a an inhibitor of a caspase activator selected from the group consisting of Calpain inhibitor 1, Calpeptin, E64, MDL28170, MG101, Acetyl-Calpastatin, PD 150606. In a preferred embodiment the apoptosis inhibitor is Boc-Asp(OMe) fluoromethyl ketone.

In another embodiment, the direct inhibition of Caspase genes and pathways may be achieved by genetic engineering of the GABAergic neurons such as by—TALENS, CRISPR-Cas9, or RNAi to promote cellular survival.

In one embodiment, the necrosis inhibitor in the kit is selected from one or more of MS-1, IM-54, GSK-872, 7-Cl-O-Nec1, Necrostatin-1, Necrosulfonamide. In a preferred embodiment, the necrosis inhibitor is Necrosulfonamide.

In another embodiment, the direct inhibition of necroptosis genes and pathways and pathways may be achieved by genetic engineering of the GABAergic neurons such as by—TALENS, CRISPR-Cas9, or RNAi to promote cellular survival.

In another embodiment, the kit of the present invention comprises one or more inhibitors of excitotoxic induced apoptosis or necroptosis selected from the group consisting of Amantadine, Memantine, Ketamine hydrochloride, pethidine, tramadol, methadone, dectropoxyphene, nitrous oxide, dextromethorphan, AP5, AP7, CPPene, Selfotel, Ethanol, Minocycline, Atomoxetine, AZD6765, Agmatine, Chlorophorm, Dextrallorphan, Dextrorphan, Diphenidine, Dizocilpine, Eticyclidine, GAcyclidine, Ketamine (other forms), Magnesium, Methoxetimine, Nitormemantine, PD-137899, Phencyclidine, Rolicyclidine, Tenocyclidine, Tiletamine, Neramexane, Elipradol, Etoxadrol, Dexoxadrol, WMS-2539, NEFA, delucemine, 8A-PDHQ, Aptiganel, HU-211, Huperzine A, Ibogaine, Remacemide, Rhynchophylline, GABApentin, Rapastinel, NRX-1074, 7-chlorkynurenic acid, 4-Chlorkyurenine, 5,7-Dichlorokynurenic acid, Kynurenic acid, TK-40, 1-Aminocyclopropanearboxylic acid, L-Phenylalanine, and Xenon; one or more inhibitors of excitability selected from Bupivacaine, Lidocaine, Cocaine, Lamotrigine, Paraldehyde, Stiripentol, Phenobarbitol, Primidone, Methylphenobarbitol, pentobarbital, Benzodiazepines (Clobazam, Clonazepam, Clozrazepate, Diazepam, Midazolam, Lorazepam, Nitrazepam, Temazepam, Nimetazepam)Potassium Bromide, Felbamate, Carboxamides (Carbamazepine, Oxcarbazepine, Esclicarbazepine) Acetate, Valproates (Valporic Acid, Sodium Valporate, Divaproex sodium, Vigabatrin, Progabide, Tiagabine, Topiramate, Pregabalin, Ethotoin, Phenytoin, Mephenytoin, Fosphenytoin, Paramethadone, Trimethadione, Ethadione, Becalamide, Primidone, Brivaracetam, Etiracetam, Levetircetem, Slectracetem, Ethosuximide, Phensuximide, Mesuximide, Acetazolamide, Sultiame, Methazolamide, Zonisamide, Pheneturide, Phenacemide, Valpromide, Valnoctamide, Perampanel, Stiripentol, Pyroxidine, Isoflurane, Levoflurane, CNV1014802, Funapide, Prilocaine, lontocaine, Levobupivacaine, Butanilicaine, Carticaine, Dibucaine, Etidocaine, Mepivacaine, Prilocaine, Trimecaine, Amylocaine, Cyclomethylcaine, alpha-Eucaine, Beta-Eucaine, Hexylcaine, Isobucaine, Piperocaine, Orhtocaine, Benzocaine, Butamibe, Chloroprocaine, Lucaine, Dimethocaine, Meprylcaine, Lucaine, Nitrocaine, Orthocaine, Propoxycaine, Novocaine, Proxymetacaine, Risocaine, Tetracaine. Raxatrigine, Tricyclic antidepressants (amitriptyline, Nortriptyline, DSP-2230, Mexilitine, Flupirtine, ziconotide; or any drug inhibiting peripheral activity including Opium and Opioids, Non-steroidal anti-inflammatories, Paracetamol, Acetenalidide, Capsaicin, Menthol, Cannabis and Cannibinoids.

In a further embodiment, the present invention provides a kit when used according to the methods of treatment as described herein.

These and other aspects of the invention are illustrated by the following non-limiting examples. It should be appreciated that in some aspects one or more embodiments described in the examples may be generally applicable in combination with one or more embodiments described above.

### EXAMPLES

### Experimental Model and Subject Details

Animals

Mice are male NOD.PRKD^{SCID.ARC} obtained from ARC (Animal Resource Centre, ARC) aged to 10 weeks and habituated to the facility and equipment for 2 weeks. All animal experiments were performed blind to treatment and assignment to treatment groups was performed pseudo randomly by an experimenter blind to behaviour data and health status. Mice were housed on a 12 hr light dark cycle and provided with standard chow and water ad libitum at all stages. All mice were maintained in a specific pathogen free facility and aseptic technique was used for all handling and experimentation. All behaviour was performed by a single male investigator. All experiments were approved by the University of Sydney Animal Ethics Committee under Animal ethics protocol 938. Experimental design and recording have been guided by the ARRIVE guidelines, and in accordance with Australian National Health and Medical Research Council guidelines.

Drosophila Stocks

Flies were reared on a standard corn meal, yeast and sucrose agar medium at 25° C. under a 12-h:12-h light:dark cycle. Canton S (BDSC 64349), painless (EP(2)2451) (BDSC 27895), ppk-Gal4 (BDSC 32078), UAS-CD8-GFP (BDSC 5130), UAS-Dcr2, UAS-tetanus toxin (active, BDSC 28838 and inactive BDSC 28839), UAS-p35 (BDSC 5072), and UAS-Lamin-GFP (BDSC 7376) flies were obtained from BDSC library. w1118 (VDRC 60000), UAS-TrpA1-RNAi (VDRC 37249), UAS-RDL-RNAi (VDRC 41101), UAS-GRD-RNAi (VDRC 5329), UAS-D-GABA-B-R1-RNAi (VDRC 101440), UAS-D-GABA-B-R2-RNAi (VDRC 110268 and VDRC 1785), UAS-D-GABA-B-R3-RNAi (VDRC 50176), UAS-LCCH3-RNAi (VDRC 37408) flies were obtained from VDRC RNAi library.

Human Pluripotent Stem Cell Line

The ATCC-BXS0116 hiPSC line was used in this study (ATCC, ACS1030).

Experimental Methods

Adult Thermal Nociception Assay System

The adult thermal nociception assay system consists of transparent polystyrene test chambers (0.3 cm height, 5.5 cm diameter clear plastic lid), a variable heat element (Model AHP-1200DCP, Part number 9-34 KB-1-0A1, of ThermoElectric Cooling America (TECA) Corp., IL, USA), a movie recording setup and behaviour analysis software. Movies were recorded with a single camera from top (Canon EOS, 700D, 18-55 mm lens); movies contain the behaviour traces of ten flies.

Fly Injury Model

The right middle leg was amputated at the femur segment using vannas scissors. Flies were 7 days old when the leg was amputated, and tested 1, 7, or 14 days later. Each set of 10 flies was lightly anesthetized on ice before being placed in a behavioural chamber. Surface was initially set at 25° C. Flies were allowed to acclimate to the test chamber, and then baseline 25° C. responses were recorded. Surface temperature was held at 25° C. for 2 mins, then raised to 30° C. for 2 min, then similarly to 35° C. for 2 min, 38° C. for 2 min, and finally at 42° C. for 1 min. A video recording camera set at 29 fps images/second and positioned above the apparatus was used to record observations of flies. Jumping behaviour was scored manually blind to the treatment using recorded videos. Speed of movement was measured using Ctrax software and packages that track individual flies. For each experiment, 3 batches of 10 flies were tested, and then results repeated with three independent groups (n=9). Statistical analysis was performed using t-test for single comparisons and ANOVA followed by a post hoc Tukey's test for multiple comparisons.

Electrophysiological Recordings

Flies were anesthetized using ice and anchored to a wax support ventral side down. Two stimulating electrodes made of tungsten connected to a stimulator (Constant Voltage Isolated Stimulator, Model DS2A-Mk.II, Digitimer) were placed into both eyes to activate the Giant Fibre System (GFS). Similarly, two tungsten stimulating electrodes were also placed in the middle of fermis segment of the right (ipsilateral) or left (contralateral) leg to activate nociceptive GFS escape through the leg. For GFS through the eye, flies were given 20 single stimuli with maximum stimulation intensity smaller than 15V. For leg stimulation nociceptive escape, the maximum stimulation intensity was less than 60V. For all experiments, stimulation duration was kept constantly at 10 μs. A tungsten ground electrode was placed into the fly abdomen. A tungsten recording electrode, sharpened in sodium hydroxide 5M (with a bench-top power supply, PSU 130-LASCAR), was placed into the left backside of the fly at the Dorsal Longitudinal Muscle fibre (DLM) to record the post-synaptic potentials (PSPs). PSPs of at least 9 flies for each group were recorded with Microelectrode AC Amplifier, Model 1800(A-M System) filtered at 0.5 kHz and digitized at 1 kHz. PSPs were analysed using AxoGraph software (AxoGraph Scientific, Berkeley, Calif.). To determine if the response measured by stimulating the leg was mediated by the central nervous system, a similar set up for recordings was used, with the head of the fly removed. Mann-Whitney Rank sum test was used to determine differences in response latency and duration.

Immunohistochemistry Studies and Imaging

Immunofluorescence on fly brains and VNCs was performed as described. Anti-GABA (A2052, SigmaAldrich) was used at dilution of 1:500, nc82 antibody (Developmental studies Hybridoma Bank) at dilution of 1:75, and cleaved caspase-3 antibody (Asp175, Cell Signaling Technology) at dilution of 1:500. Secondary antibodies (Alexa 488, Alexa 555 and Alexa 647, from Thermo Fisher) were used at dilutions of 1/500. Confocal sections were acquired using Leica DMI 6000 SP8 confocal microscope with 40×NA 1.30 oil objective at 0.6 μm intervals. Top-view pictures were made by performing maximum projections of image stacks in ImageJ (NIH; http://rsbweb.nih.gov/ij/); and tangential side view images were made by using ImageJ and Leica Application Suite X, LASX software. GABA foci were quantified using 3D-object counter function in ImageJ. Leg imaging was performed at 16×/0.5 IMM objective at 2.34 μm intervals and tarsus segment imaging was acquired at 40× oil objective at 0.6 μm intervals, of the same confocal microscope. Neuropathy of ppk+ neurons in the leg was assessed by measuring dendritic length retained in the leg using ImageJ.

HiPSC Culture and Differentiation into GABA Interneurons or Sensory Neurons

HiPSC were maintained on matrigel coated surfaces in mTESR1 media (Stem Cell technologies).

HiPSC were differentiated in GABA interneurons using an adapted version of the protocol described by Kim T G et al., Stem Cells, 2014. Briefly, hiPSC were dissociated with TripLE (Invitrogen) and grown for 2 weeks in suspension into ultra-low attachment binding plates to allow the formation of embryoid bodies (EBs). For neural induction, cells were treated with SMAD inhibitors LDN193189 (100 nM, day 0 to day 14) and SB431542 (10 μM, day Oto day 10). For MGE (Medial Glanglionic eminence) induction, cells were treated with Wnt inhibitor IWP2 (5 μM, day 0 to day 7), SHH activator SAG (Smoothened Agonist—0.1 μM, day 0 to day 21) and growth factor FGF8 (100 ng/mL, day 8 to day 21). Rock inhibitor is added on the first day of differentiation only. After 2 weeks in suspension, EBs were transferred to polyornithine (PLO) and fibronectin (FN) coated surfaces. At day 21, EBs were dissociated and cells were replated on PLO/FN coated plates on differentiation media containing 10 ng/mL BDNF, 10 ng/mL GDNF and 2.5 μM gamma secretase inhibitor DAPT for further differentiation and maturation.

HiPSC were differentiated in sensory neurons following the protocol described in Young G T et al., Molecular Therapy, 2014, with the exception that mitomycin C was omitted.

Cells Preparation for Transplantation

HiPSC-derived GABA interneurons were dissociated at 25 days of differentiation and resuspended to a final concentration of 100,000 cells per microliter in injection media made of Hank's balanced salt solution (HBSS) with 10 ng/mL GDNF, 20 μM Boc-Asp(OMe) fluoromethyl ketone (Broad spectrum caspase inhibitor—apoptosis inhibitor) and 50 nM Necrosulfonamide (a MLKL inhibitor—necrosis inhibitor).

Open Field

Mice were habituated in their home cages to an evenly lit darkened room with two sources of upward illumination. The light in each corner of a box was measured by a mobile phone light meter (Motorola). Mouse behaviour was recorded for five minutes on each test occasion. The behaviour was then analysed by Topscan behaviour analysis software.

SNI Modified Basso Mouse Scale

A modified form of the Basso was used to assess gait following transplantation. Briefly mice were scored for ankle movement (out of 3, never=0, sometimes=1, mostly=2, always=3) plantar stepping (out of 3, never=0, sometimes=1, mostly=2, always=3) Coordination (out of 3, never=0, sometimes=1, mostly=2, always=3) and rotation of the paw (parallel=2, rotated=1, not walking=0). Mice were scored bilaterally from videos.

Von Frey

Mice were habituated on 3 separate days. For the baseline assessment of pain, mice were then tested on 3 different days. The baseline threshold is defined as the average of the threshold for the last two days of testing. Von Frey filaments were applied to the sural portion of the footpad and applied 10 times at each stimulus threshold (0.04 to 2.0 g). The response to each filament was recorded until 100% response was reached. Thresholds are reported as the lowest filament causing responses in 50% of tests. Mice were assessed 6 days after spared nerve injury and then weekly.

Acetone

A drop of acetone was applied to the mouse hind paw using ejection of around 30 ul from a 1 ml insulin syringe. The time spent licking and biting for one minute was recorded.

Spared Nerve Injury

Mice were anaesthetised with Isoflurane with 3% at 0.8 L/min Oxygen for Induction followed by 2% isoflurane for maintenance. Depth of anaesthesia was confirmed by absence of toe reflexes and palpebral reflex. Eyes were provided with lubrication to prevent damage to the corneal. The skin was incised with a scalpel and the muscle was blunt dissected to isolate the left sciatic nerve. The tibial and common peroneal portions of the nerve were ligated with Ethilon 4-0 nylon suture material (Johnson and Johnson International) and a portion of nerve was removed taking care not to touch the sural nerve. The incision was then closed with wound clips (Reflex, FineScience Tools). NOD SCID Mice were provided with 2.5 mg/kg enrofloxacin (Baytril, Bayer) in normal saline 0.9% (Pfizer) daily by subcutaneous injection for 7 days. Motor behaviour was assessed after 4-5 days and pain behaviour was assessed at 6 days.

In Vivo Laminectomy and Spinal Cord Cell Injection

Mice were anaesthetised with a Ketamine (Ketamil)/Xylazine cocktail (100 mg/8 mg/kg) by intraperitoneal injection. Anaesthetic depth is regularly monitored by toe pinch and absence of palpebral reflexes. Breathing rate, body temperature and mucous membrane reperfusion were also monitored. Body temperature was maintained with a 37° C. heating plate and Oxygen 0.8 to 1 L/min was provided during the procedures to prevent hypothermia and hypoxia respectively. An incision is made along the back and L1 is removed by careful pedicular dissection using ophthalmic vannas spring scissors (World Precision instruments). A 2 μl injection targeting the lumbar dorsal horn was made using a stereotaxic apparatus (Kopf) and a Hamilton Syringe with a custom designed needle (29 gauge with point style 4 and 30° Angle, 1.5-inch length) ipsilateral to the injury using the posterior central spinal artery as a landmark. Briefly the needle was advanced until the dura was initially punctured then lowered using the digital stereotaxic monitoring device. 2 μl of solution was injected slowly and left in place for 5 minutes to prevent efflux. The superficial fascia was sutured using Vicryl 5-0 Reverse cutting sutures (Johnson and Johnson) and the incision was closed with 2-3 wound clips. Bupivacaine 8 mg/kg (Pfizer) was injected and irrigated subcutaneously/cutaneously around the wound edges and Enrofloxacin was provided daily for 10 days. Pain behaviour was first assessed 6 days following surgery. Mice were monitored on a daily basis for the duration of the experiments. Mice were provided with 40° C. warmed normal saline (Pfizer) immediately following the procedure. Mice were monitored every 2 hours post procedure and provided with warmth until full recovery.

Perfusion

Deeply anaesthetised mice were taken at four weeks following nerve injury. The chest was opened and the heart was exposed and a winged catheter (Griener Bio-one) was inserted into the left ventricle. Mice were perfused by 25 ml 0.1M Phosphate buffer (PB, pH 7.4, Sigma) followed by 25 ml 4% (w/v) Paraformaldehyde (PFA, Sigma) in 0.1M PB using a syringe driver. Spinal tissue was removed at least 2 mm rostrally and caudally to the injection site Tissue was post fixed in PFA for 2-4 hours and then cryoprotected overnight in 30% (w/v) sucrose. The resultant tissue was cut to fit cryomolds and flash frozen embedded in O.C.T (VWR). Spinal cords were sectioned at 16-20 μm on a cryostat (Thermo Fisher).

Antibody Staining

Cells were fixed with 10% formalin (Sigma-Aldrich, HT5011) for 15 min at room temperature and were stained for specific neuronal markers using the listed antibodies and dilutions. Appropriate Alexa Fluor secondary antibodies (Thermo Fisher Scientific Life Sciences; 1:500) were used, and nuclei were visualized with Hoechst 33342 (1:5,000; Thermo Fisher Scientific Life Sciences catalog no. H3570).

For spinal cord staining, cryosections were allowed to equilibrate to room temperature for 20 minutes. Sections were then rinsed three times in PBS for five minutes. Slides were blocked for 1 hour in blocking solution at room temperature (PBS, Bovine Serum Albumin (2%) and 0.3% Triton X100). Primary antibodies were added diluted in blocking solution and incubated overnight at 4° C. in a humidified chamber. Appropriate secondary antibodies were added at the dilutions listed. Finally, the slides were washed 3 times in PBS for 15 minutes each and the slides were coverslipped with Vectashield antifade mounting solution.

RNA Extraction

Total RNA was harvested from hiPSC and GABAergic neuron cultures by using the Favorgen Blood/Cultured Cell Total RNA kit (Favorgen Biotech Corp.). RNA was treated with DNase in solution using On-Column DNase I Digestion Set (Sigma) and maintained with Ribosafe RNase inhibitor (Bioline) 40 U per sample. Quality and quantity of RNA were assessed by absorbance spectroscopy (Nanodrop, Thermo Fisher Scientific Life Sciences), Agilent Bio-analyser and Agarose gel electrophoresis.

qPCR

After quantification, 1 μg of RNA was retrotranscribed with Superscript III First-Strand Synthesis SuperMix for qRT-PCR (Thermo Fisher Scientific Life Sciences). A total of 2 μl of cDNA was used for qPCR using the SYBR Select Master Mix. Real-Time PCR was run on a LightCycler 480 Instrument II (Roche Life Science). The cycling program for all genes is the following:
1. 95° C. 10′ Initial denaturation step
2. 95° C. 30″
3. 58° C. 30″
4. 72° C. 30″ go to 2 for 35 cycles
5. Melting curve analysis.

Primer sequences are as follows:

GAD1 FWD: CACAAGGCGACTCTTCTCTTC
GAD1 RVR: GCGGACCCCAATACCACTAAC
GAD2 FWD: TTTTGGTCTTTCGGGTCGGAA
GAD2 RVR: TTCTCGGCGTCTCCGTAGAG
VGAT FWD: ACGTCCGTGTCCAACAAGTC
VGAT RVR: AAAGTCGAGGTCGTCGCAAT
SST FWD ACCCAACCAGACGGAGAATGA
SST RVR GCCGGGTTTGAGTTAGCAGA
SOX 6 FWD: GGATGCAATGACCCAGGATTT
SOX6 RVR: GGATGCAATGACCCAGGATTT
Catrenin FWD: ACTTTGACGCAGACGGAAATG
Caltrenin RVR: GAAGTTCTCTTCGGTTGGCAG
Calbindin FWD: GGCTCCATTTCGACGCTGA
Calbindin RVR: GCCCATACTGATCCACAAAAGTT
TUBB3 FWD: GGCCAAGGGTCACTACACG
TUBB3 RVR: GCAGTCGCAGTTTTCACACTC
LHX6 FWD: GGGCGCGTCATAAAAAGCAC
LHX6 RVR: TGAACGGGGTGTAGTGGATGT
OLIG2 FWD: CCAGAGCCCGATGACCTTTTT
OLIG2 RVR: CACTGCCTCCTAGCTTGTCC
NKX2.1 FWD: AGCACACGACTCCGTTCTC
NKX2.1 RVR: GCCCACTTTCTTGTAGCTTTCC
PAX6 FWD: TGGGCAGGTATTACGAGACTG
PAX6 RVR: ACTCCCGCTTATACTGGGCTA

RNA Sequencing

Novogene performed RNA Sequencing according to their standard in house methods. Library preparation was performed using the NEBNext Ultra RNA library prep kit for Illumina. Index coded samples were clustered using the HiSeq PE Cluster Kit cBot-HS (illumina) and the resultant libraries were sequenced on an illumine hiseq platform and 125/150BP paired end reads were generated.

RNA Sequencing Analysis

Clean reads were obtained by removing reads containing adapter, poly-N and low quality reads. All downstream data analysis was performed on clean data. An index of the reference genome was produced using Bowtie v2.2.3 and paired end clean read were aligned to the reference genome using TopHat v2.0.12. HTSeq v0.6.1 was used to count read numbers mapped to each gene and then FPKM was calculated based off gene length and the number of read counts mapped to the gene. Raw read counts were inputted into DESeq2 package in R and differential expression was assessed. For visualization heatmaps FPKM was normalized to the largest value FPKM. The resulting p values were adjusted by Benjamani Hochberg and genes with an adjusted p value of less than 0.05 were assessed as differentially expressed.

Flow Cytometry Analysis

Cells were dissociated with TryPLE (Invitrogen), fixed in 10% Formalin (Sigma-Aldrich, HT5011) for 20 min and incubated with blocking buffer (PBS with 3% BSA and 0.3% Triton X100). Blocked cells were incubated with primary antibody (conjugated anti-β3 Tubulin antibody-Alexa Fluor 488, Abcam or anti-GAD65 antibody, Abcam) for 30 min in blocking buffer. Cells were washed 3 times with PBS and incubated with Alexa Fluor 594-conjugated secondary antibody (Abcam) for another 30 min. After washing with PBS, cells were resuspended in PBS with 3% BSA and analyzed. A mouse IgG isotype control antibody for GAD-65 was used under the same conditions. Unlabelled controls were also used as controls. Acquisition of 10,000 events was collected using FACSAria (BD Biosciences). Flowjo software was used to analyze raw data.

ELISA

At 25 DIV GABAergic neurons were left in media for three days. The media was aspirated and tested using a Rat Gamma-Amino Butyric Acid ELISA (MyBioSource). Absorbance values were transformed according to the manufacturer's instructions using a 4 point logistic regression using GraphPad Prism.

GABA Vs Induced Pluripotent Stem Cell Proteomics

GABA neurons were derived by the methods described herein. At 25 DIV they were lysed by gentle washing in ice cold phosphate buffered Saline 3 times. Denaturing lysis buffer (4% SDS, 20 mM Sodium phosphate 6.0, 100 mM NaCl, complete protease inhibitor (EDTA Free, Roche), 10 mM NaF, 10 mM Sodium Pyrophosphate, 2 mM sodium orthovanadate, 60 mM B-Glycerophosphate) was added to the well and they were scraped using a cell scraper, samples were heated for 10 minutes at 65° C. Samples were then sonicated (30 second sonication ON/OFF cycling, 10 minutes total sonication, 80% amplitude at 18° C.) on a Q.Sonica 800.

Protein digestion, peptide clean-up and quantitation—Proteins (100 ug) were reduced by the addition of triscarboxyethylphosphine (TCEP) to a final concentration of 10 mM. The protein samples were heated to 65° C. in a ThermoMixer-C(Eppendorf) for 10 min at 1000 rpm. Once cooled to room temperature, N-ethylmaleimide (NEM) was added to the fractions at a final concentration of 20 mM and allowed to incubate for 30 min at room temperature. The fractions were buffer exchanged and trypsin digested using the SP3 method described previously (Ultrasensitive proteome analysis using paramagnetic bead technology. Hughes C S, Foehr S, Garfield D A, Furlong E E, Steinmetz L M, Krijgsveld J. Mol Syst Biol. 2014 Oct. 30; 10:757. doi: 10.15252/msb.20145625).

LC-MS/MS and Analysis of Spectra

Using a Thermo Fisher Scientific EasyLC 1000 UHPLC, peptides in 4% (vol/vol) formic acid (injection volume 3 μL, approximately 1000 ng peptides) were directly injected onto a 50 cm x 75 μm reverse phase C18 column with 1.9 μm particles (Dr. Maisch GmbH) with integrated emitter. Peptides were separated over a gradient from 5% acetonitrile to 30% acetonitrile over 90 min with a flow rate of 300 nL min-1. The peptides were ionized by electrospray ionization at +2.3 kV. Tandem mass spectrometry analysis was carried out on a Q-Exactive mass spectrometer (Thermo Fisher Scientific) using HCD fragmentation. The data-dependent acquisition method used acquired MS/MS spectra on the top 20 most abundant ions at any one point during the gradient. All the RAW MS data have been deposited to the ProteomeXchange Consortium (http://proteomecentral.proteomexchange.org) via the PRIDE partner repository with the dataset identifier PXDOOXXXX, username: reviewerXXXX@ebi.ac.uk, password: XXXXX. The RAW data produced by the mass spectrometer were analysed using MaxQuant. Peptide and Protein level identification were both set to a false discovery rate of 1% using a target-decoy based strategy. The database supplied to the search engine for peptide identifications was the Human Swissprot database downloaded on the 30 Sep. 2017. The mass tolerance was set to 4.5 ppm for precursor ions and MS/MS mass tolerance was set at 20 ppm. Enzyme was set to trypsin (cleavage C-terminal to R/K) with up to 2 missed cleavages. Deamidation of N/Q, oxidation of M, N-terminal pyro-E/Q, protein N-terminal acetylation, were set as variable modifications. N-ethylmaleimide on C was searched as a fixed modification. The output from MaxQuant has also been uploaded to the ProteomeXchange Consortium under the same identifier given above.

Protein Assay

Sample protein concentration was determined using a bicinchoninic acid protein assay, according to the manufacturer's instructions (Thermo Scientific).

Western Blots

10 μg of extracted human stem cell proteins and ladder (SeeBlue Plus 2 Prestained, ThermoFisher Cat #. LC5925) were electrophoresed (1 hour, 150V) on 8-12% (w/v) SDS-polyacrylamide gels (S5). Separated proteins were then wet transferred to nitrocellulose membranes (Li-Cor) at 35V for 1.5 hours and then blocked in 5% (w/v) dried skimmed milk in PBS and incubated in primary antibodies dissolved in 5% BSA, 0.1% PBS Tween-20 and 0.02% sodium azide blocking buffer (TableXXX), overnight at 4° C. After washing, nitrocellulose membranes were incubated with the appropriate fluorophore-conjugated secondary antibodies (see table). Blots were visualized using an Odyssey® infrared imaging system (Li-Cor Biosciences). For Coomassie stains (InstaBlue Commassie, Sigma Aldrich, Cat #. ISB1L) 10 μg of protein was electrophoresed at 150V for 1 hour alongside fibronectin and matrigel controls and incubated overnight at RT in Coomassie stain. Following 2 hours of destaining, the gels were imaged using Odyssey® infrared imaging system.

Data Analysis

Proteomics data was analysed by Student's t-test and adjusted for False discovery with the Benjamani Hochberg procedure.

### Example 1. Neuropathic “Pain” is a Conserved Response to Injury

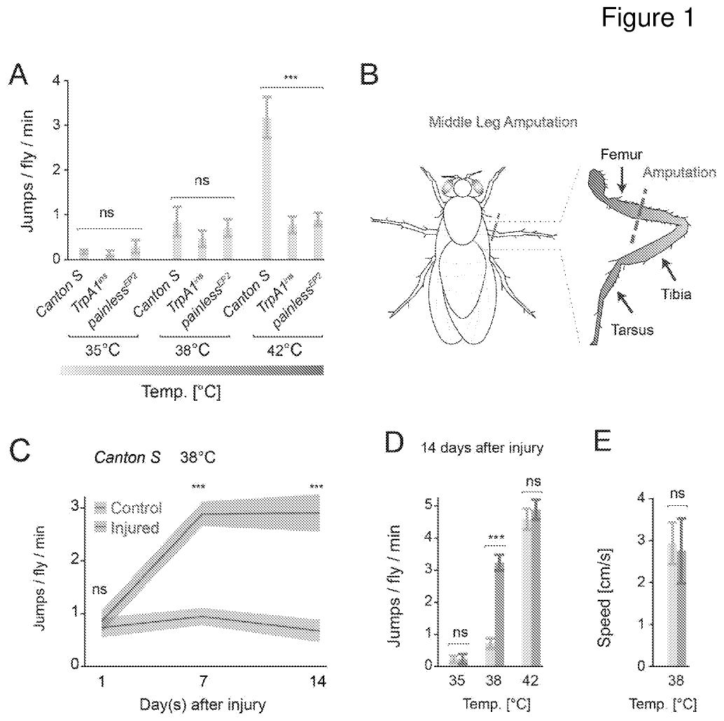

To investigate chronic “pain” in a genetically tractable invertebrate, the inventors established a nerve injury model in the adult fruit fly. In flies, surface temperatures>42° C. trigger a strong nociceptive avoidance response or death within minutes. Exploiting this behaviour, the inventors developed a fly hot plate escape paradigm to investigate nociceptive thresholds. Wild-type (Canton S) fruit flies showed minimal escape responses when the surface was set from 25-38° C. (FIG. 1A, and not shown). However, when animals were exposed to noxious heat (42° C.), uninjured flies showed a robust nociceptive escape response with animals exhibiting ˜3 escape responses/fly/minute (FIG. 1A). Since Drosophila TrpA family members TrpA1 (Neely et al., 2011; Zhong et al., 2012) and painless (Neely et al., 2010; Tracey et al., 2003) are required for acute heat nociception in larvae and adult flies, the inventors tested if these receptors are also involved in this response. Indeed, both TrpA1 and painless were required for acute escape behaviour at the noxious (42° C.) temperature (FIG. 1A).

The inventors next injured flies and asked if injury altered the thermal escape response profile. The inventors amputated the right middle leg of wild type animals (FIG. 1B), allowed the animals to recover, then evaluated escape responses at different temperatures. While intact animals displayed minimal escape attempts when exposed to a 38° C. surface, after amputation flies showed significantly more escape behaviours (FIG. 1C). This response was absent immediately after injury, first became apparent ˜7 days after injury, and persisted past 14 days (FIG. 1C, Figure S1A and B). Injury did not alter escape responses at the noxious temperature (42° C.), which would be considered a hyperalgesic response, but was limited to subnoxious sensitisation (38° C.), consistent with thermal allodynia; where a “painful” behavioural response is elicited by an innocuous stimulus (FIG. 1D, Figure S1A and B). Analysis of overall velocity showed no gross change in movement after limb amputation, indicating the phenotypes observed are not due to generalised differences in activity (FIG. 1E). Together, these data show that fruit flies exhibit allodynia in response to serious injury.

### Example 2. Allodynia is Mediated by TrpA1 in ppk+ Sensory Neurons

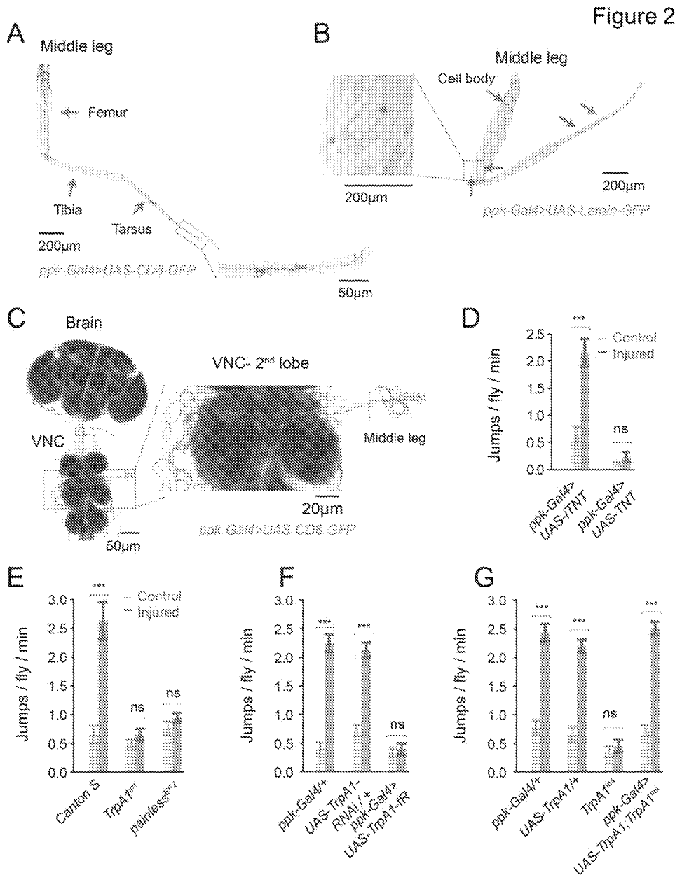

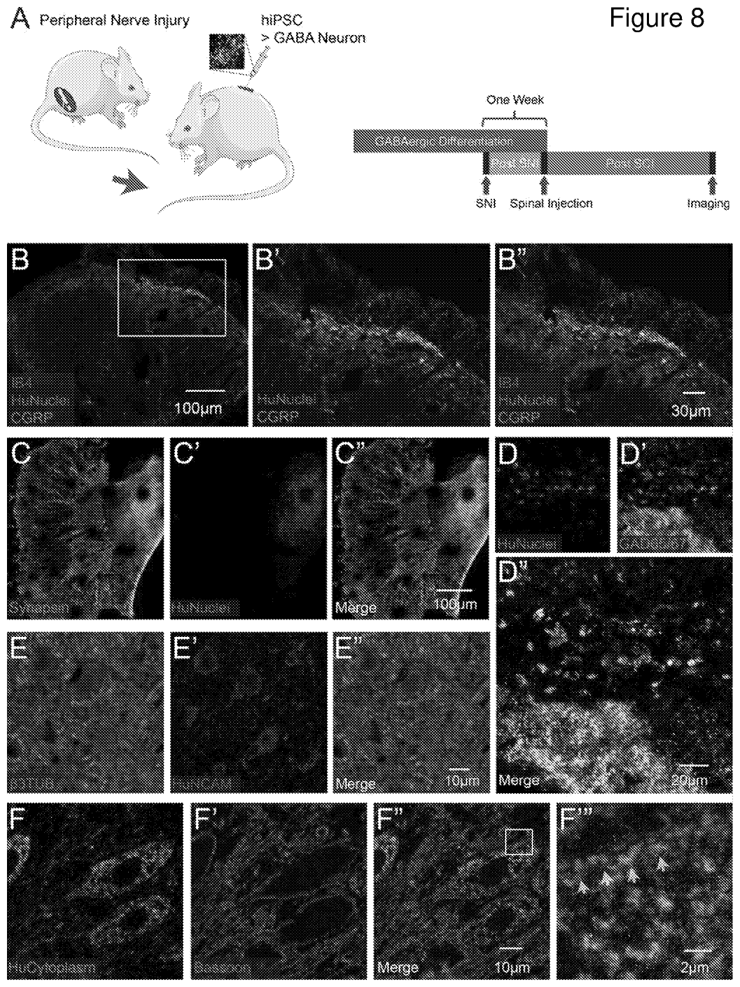

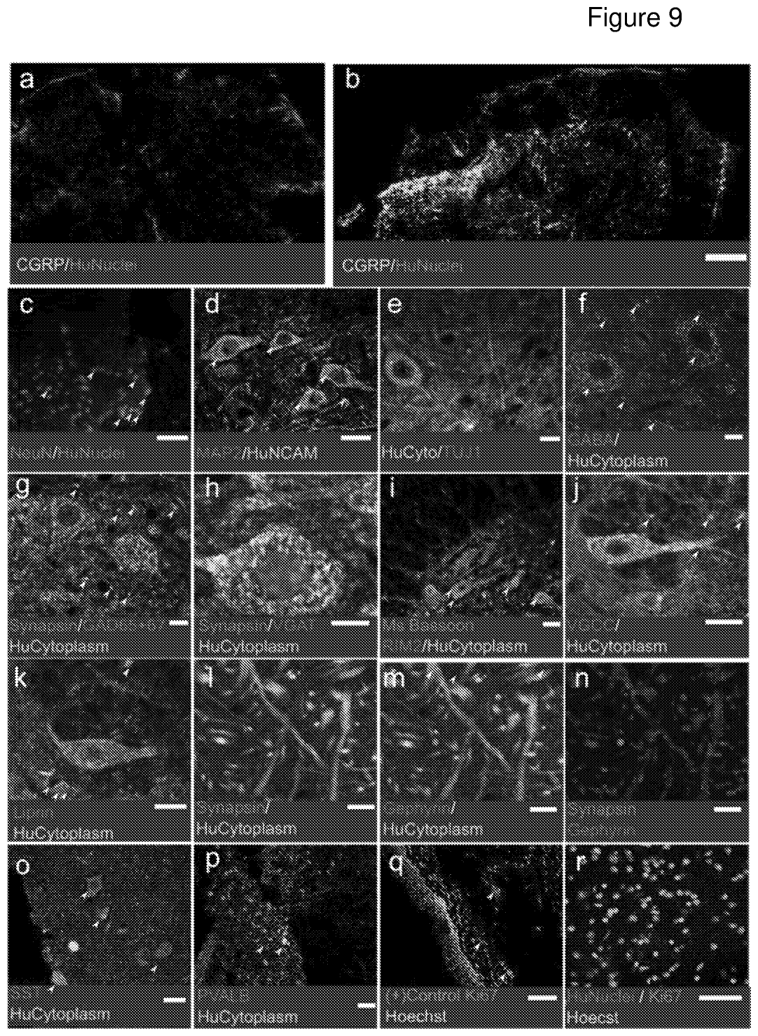

In larvae, ppk+ sensory neurons tile the body of the animal and transduce acute noxious heat responses (Zhong et al., 2010). In the adult fly the inventors observed ppk+ neurons organised into likely sensory structures in the leg (FIG. 2A), with ppk+ cell bodies situated along the leg (FIG. 2B) and ppk+ neurons send projections both peripherally and toward the ventral nerve cord (VNC) and brain (FIG. 2C, FIG. 8C-E). Importantly, when the inventors blocked synaptic output from ppk+ neurons with UAS-tetanus toxin, animals no longer exhibited allodynia after injury (FIG. 2D) but showed otherwise comparable mobility (not shown). Moreover, while control animals exhibited a sensitised escape response to 38° C. after injury, both painless and TrpA1 mutant animals were completely resistant to this effect (FIG. 2E) and did not even show sensitisation at 42° C. Finally, driving TrpA1 RNAi in ppk+ sensory neurons was sufficient to block allodynia (FIG. 2F), and sensitisation was completely rescued by re-introducing TrpA1 specifically in ppk+ sensory neurons on a TrpA1 mutant background (FIG. 2G). Thus, in fly, neuropathic allodynia requires the conserved nociceptive TRP channel TrpA1 expressed specifically in ppk+ nociceptive sensory neurons.

### Example 3. Peripheral Neuropathic Injury Causes Allodynia Via a Central Mechanism

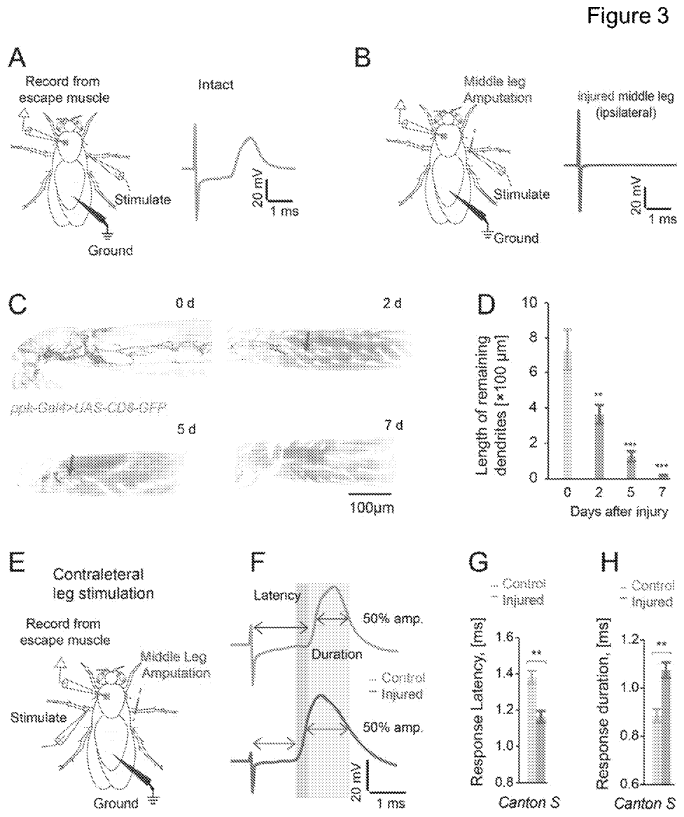

Because flies exhibit a “jumping” escape response when placed on a hot surface, and this response shows sensitisation after injury, the inventors next investigated if activating sensory neurons in the leg could directly trigger the escape response circuit. The inventors stimulated nociceptive sensilla on the middle leg of the intact fly, and evaluated the escape response by intracellular recording from the Dorsal Longitudinal Muscle (DLM), the final step in the Drosophila escape response circuit (FIG. 3A). Stimulation of the intact leg triggered a robust escape response (FIG. 3A). The giant fibre response can occur without participation from the brain (FIG. 9A). However, the inventors found leg stimulation leading to an escape response was not a local reflex but required higher order brain function (FIG. 9B). Intriguingly, while amputation of the middle leg caused behavioural sensitisation to innocuous heat, when the inventors directly stimulated the injured leg the inventors observed no response (FIG. 3B). In accordance, the inventors saw a gradual neuropathy of proximal ppk+ sensory neurons in the injured leg over 7 days (FIG. 3C, quantified in D), and a similar loss of degeneration of axotomised neurons is observed after peripheral nerve transection in mammals (Tandrup et al., 2000).

Since the remaining section of the injured leg shows severe sensory neuropathy and was unresponsive to stimulation, the inventors instead stimulated the contralateral uninjured leg of amputated flies and assessed activation of the escape response (FIG. 3E). Strikingly, 7 days after injury the inventors observed clear changes when stimulating the contralateral leg, with the overall escape response velocity occurring 0.2 ms faster (FIG. 3F quantified in G) and the response duration persisting 0.2 ms longer (FIG. 3F quantified in H). Changes after injury occurred in both ascending and descending components of this nociceptive escape circuit, since when the inventors bypassed the afferent sensory input and directly stimulated the descending escape circuit the inventors still observed 50% of the total enhanced response velocity (0.1 ms faster; FIG. 9C). Together, these data show that peripheral injury leads to sensory neuropathy, contralateral sensitisation, and experience-driven plasticity of the nociceptive escape circuit.

### Example 4. Central Loss of GABAergic Inhibition Causes Neuropathic Allodynia

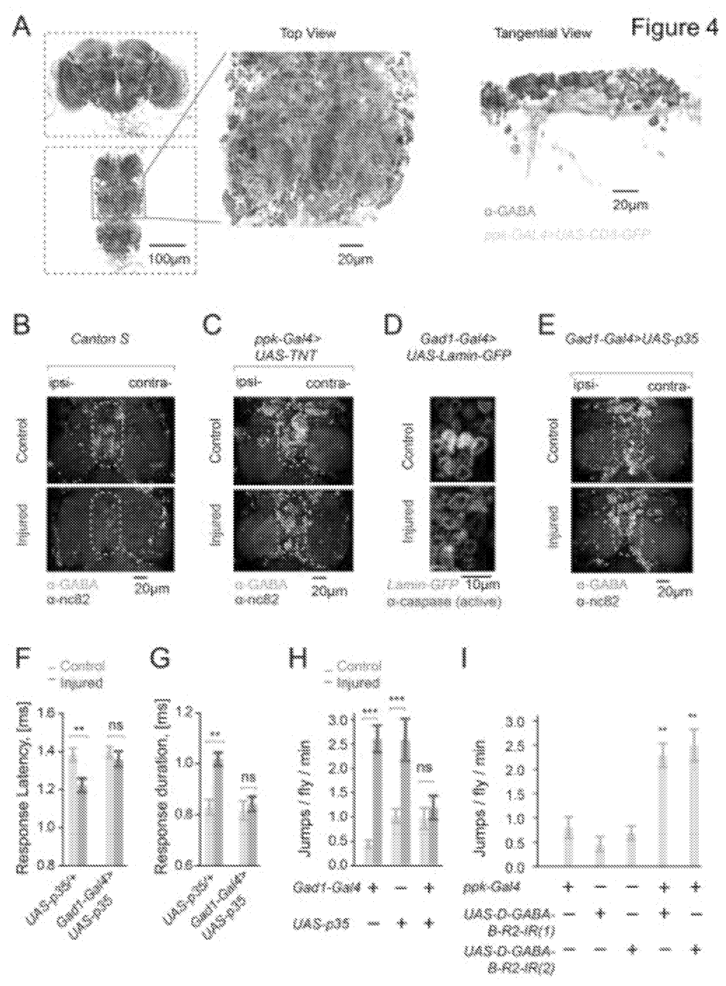

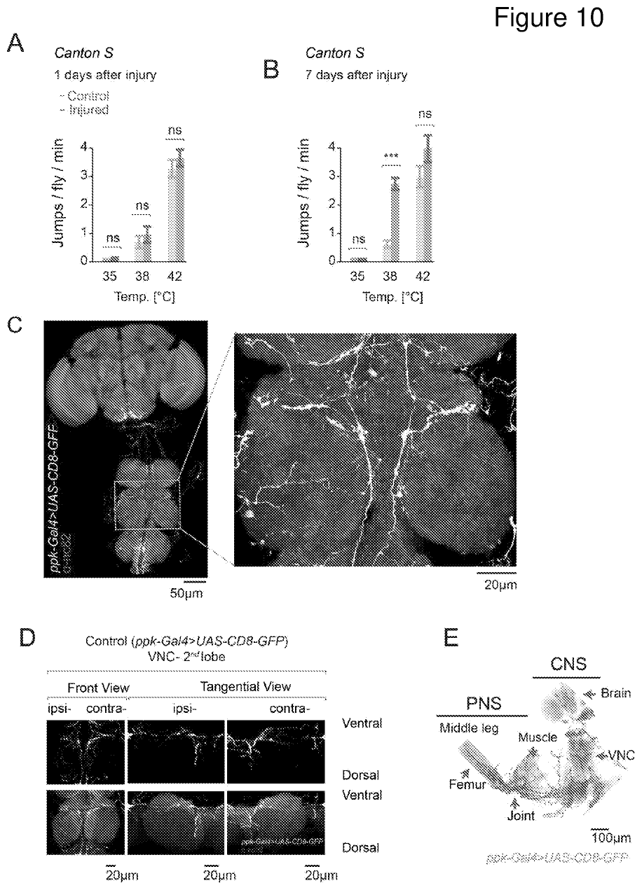

ppk+ sensory neurons project from the leg into the ventral “horn” of the Drosophila CNS (FIG. 2A-C, FIG. 8C-E). By co-labelling nociceptive (ppk+) and GABAergic neurons the inventors observed a close interaction between these two populations in the VNC. Importantly, 7 days after injury, the inventors observed a dramatic ˜40% reduction in GABA immunoreactivity in both the ipsilateral and contralateral sections of the 2^{nd} VNC lobe (FIG. 4A-B; top view, FIG. 9D; transverse plane, quantified in 9E), with this loss primarily occurring along the VNC midline. A significant yet less severe reduction in GABA foci also occurred in the first and third lobes of the VNC (FIG. 10A-B), however no difference in the number of GABA foci was observed in the brain of injured animals (FIG. 10C), i.e. loss of GABA immunoreactivity was localised to the VNC. Loss of GABAergic neurons was not due to direct damage of these cells, since no GABAergic nuclei or projections were observed in the fly leg (not shown). TrpA1 was not required for loss of GABAergic foci (not shown), however blocking synaptic output from ppk+ sensory neurons (ppk-Gal4 driving UAS-tetanus toxin) completely prevented loss of VNC GABA foci (FIG. 4C quantified in 9F) confirming the neuropathic nature of this injury.

Since pharmacological inhibition of caspase can prevent GABA loss and suppress the generation of neuropathic pain in rodents (Scholz et al., 2005), the inventors assessed a role for caspase in regulating central GABA in the fly. Intact animals showed little GABA/active caspase co-labelling in the VNC (GABAergic neurons labelled by Gad1-Gal4>>UAS-Lamin-GFP; FIG. 4D quantified in 10E). After injury the total number of GABAergic nuclei in the VNC was drastically reduced (quantified in 10D), while the number of Gad1/active caspase double positive cells significantly increased (FIG. 4D; quantified in 10E). To directly test if GABAergic cell loss is a caspase-dependent event, the inventors drove expression of the caspase inhibitor p35 specifically in GABAergic neurons (Gad1-Gal4>UAS-p35). While blocking caspase did not alter the baseline number of GABA foci in the fly VNC, ectopic expression of p35 in Gad1+GABAergic neurons completely blocked loss of GABA foci after nerve injury (FIG. 4E, quantified in 10F). The inventors next tested if blocking GABAergic (+) cell loss had functional consequences on the overall nociceptive circuit in injured animals. While the nociceptive escape circuit showed enhanced response latency and duration in the parental control line (UAS-p35/+), suppressing GABAergic cell death (Gad1-Gal4>UAS-p35) completely blocked all changes in the nociceptive escape circuit after injury (FIG. 4F-G). Importantly, parental control lines exhibited neuropathic allodynia after leg amputation, whereas blocking caspase-mediated GABAergic cell death completely suppressed this response (FIG. 4H). Conversely, nociceptor specific (ppk-Gal4) RNAi knockdown of the metabotropic GABA-B-R2 or the ionotropic GABA/Glycine receptor subunit Resistant to dieldrin (Rdl) could promote allodynia and enhance escape behaviour in response to subnoxious temperature (38° C.) in uninjured animals (FIG. 41, FIG. 10G). Together, these data show that in the fly, loss of central GABA is necessary and sufficient for thermal allodynia.

### Example 5. iPSC-Derived GABAergic Transplants can Therapeutically Treat Neuropathic Pain

The inventors' fly neuropathic studies highlight loss of central inhibition as a core underlying pathology driving neuropathic pain. Similarly, in mammalian pain perception, inhibitory interneurons that produce GABA play an important role in the central gating of pain in the spinal cord. To this end, the inventors developed a preclinical GABAergic transplant protocol to assess therapeutic viability of this approach. Cell replacement therapy would require the transplantation of autologous material so the inventors investigated the potential utility of human induced pluripotent stem cells (hiPSC).

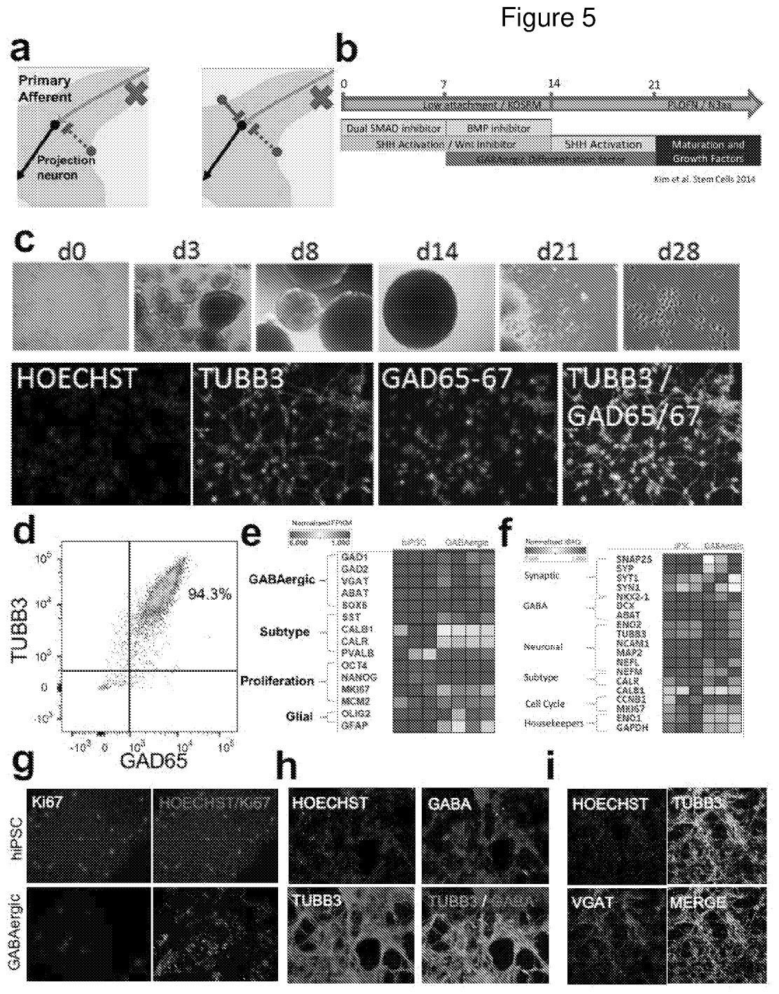

GABAergic neurons were differentiated in vitro from hiPSC through a protocol as hereinbefore described (shown in FIG. 5B). hiPSC are dissociated with TripLE (Invitrogen) and grown for 2 weeks in suspension into ultra-low attachment binding plates to allow the formation of embryoid bodies (EBs). For neural induction, cells are treated with SMAD inhibitors LDN193189 (100 nM, day 0 to day 14) and SB431542 (10 μM, day 0 to day 10). For MGE (Medial Glanglionic eminence) induction, cells were treated with Wnt inhibitor IWP2 (5 μM, day 0 to day 7), SHH activator SAG (Smoothened Agonist—0.1 μM, day 0 to day 21) and growth factor FGF8 (100 ng/mL, day 8 to day 21). Rock inhibitor is added on the first day of differentiation. After 2 weeks in suspension, EBs are transferred to polyornithine (PLO) and fibronectin (FN) coated surfaces. At day 21, EBs were dissociated and cells were replated on PLO/FN coated plates on differentiation media containing 10 ng/mL BDNF, 10 ng/mL GDNF and 2.5 μM gamma secretase inhibitor DAPT for further differentiation and maturation. The differentiation protocol efficiently drove the differentiation of hiPSC to GABAergic (GAD65/67+) neurons (TUBB3+) within 28 days (FIG. 5C).

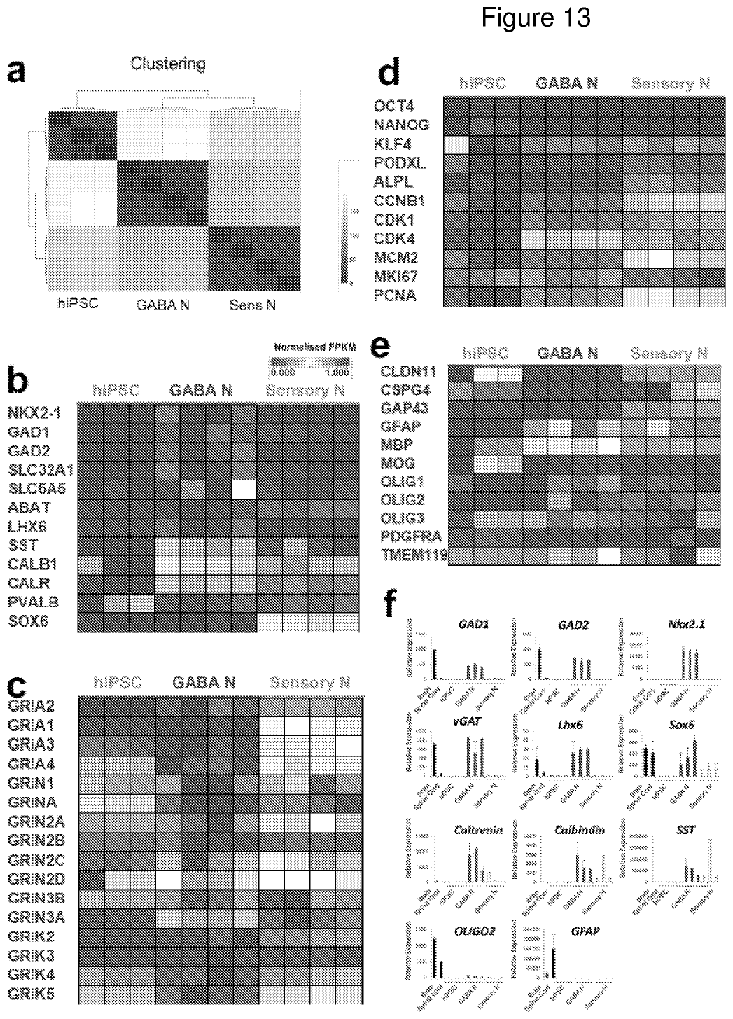

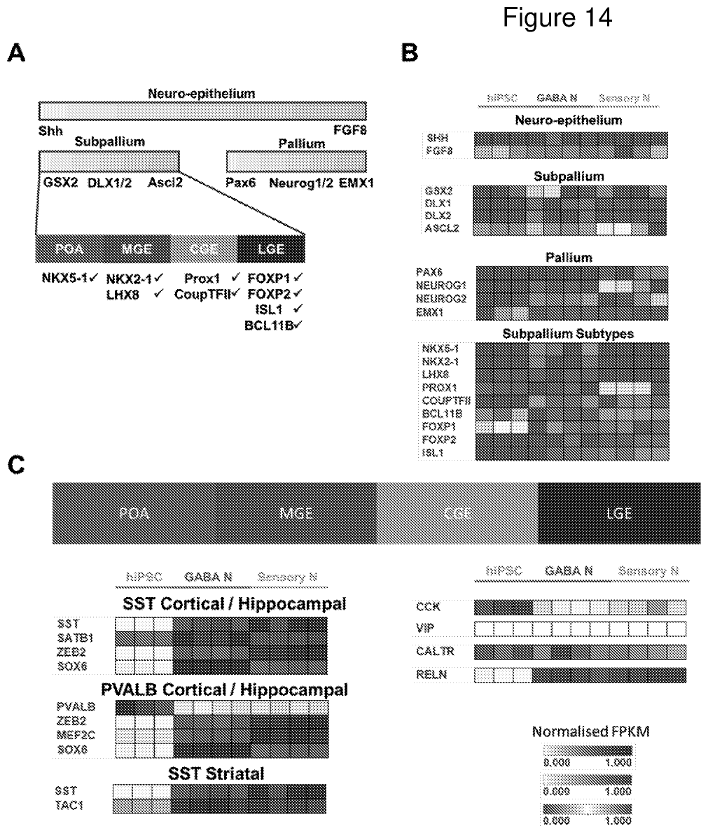

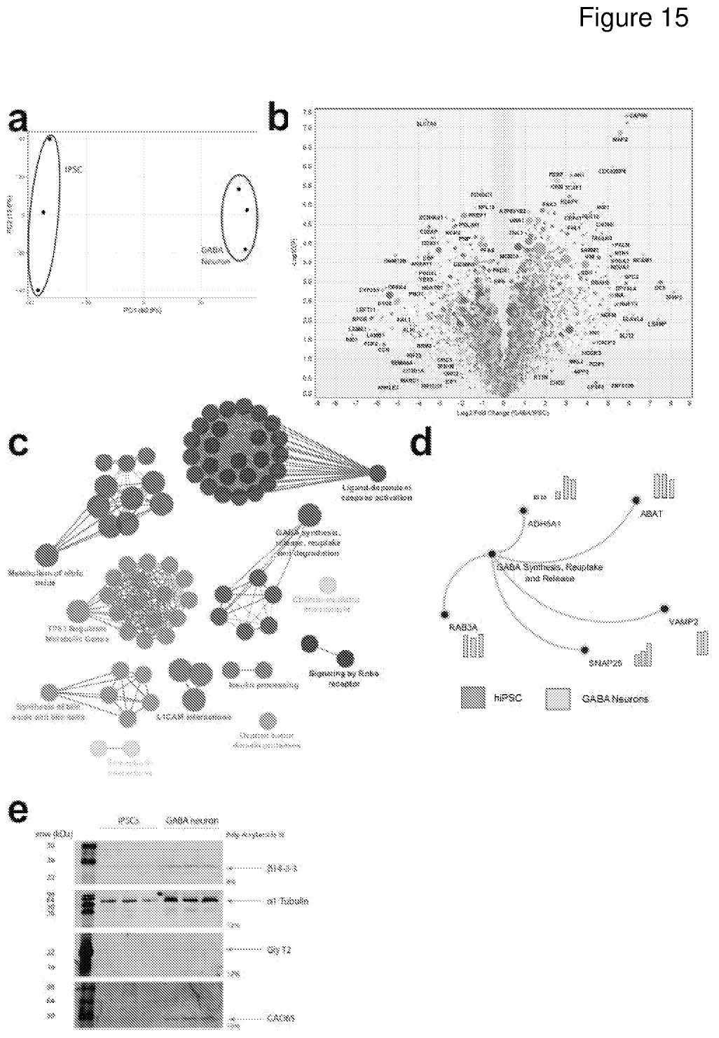

By FACS, differentiation conditions promoted an overall shift in the TUBB3 expression profile and a pure population of GAD65+ cells (-95% pure, FIG. 5D). To further characterize the molecular profile of the transplant materials, RNA sequencing was performed in DIV25 hiPSC-derived GABAergic neurons as well as the parental hiPSC. To confirm GABAergic specificity, the transcriptomes of hiPSC-derived sensory neurons were also analysed. Differentiated GABAergic neurons expressed GABA-specific transcripts and somatostatin subtype markers (FIG. 5E, FIG. 13B). HiPSC-derived GABAergic neurons also downregulated proliferation or pluripotency markers (FIG. 5E, FIG. 13D), upregulated glutamate receptors (FIG. 13C), and expressed OLIG2, likely due to undifferentiated precursor cells (FIG. 5E, FIG. 13E). Differentiated GAB Aergic neurons primarily exhibit a subpallium MGE and CGE differentiation state most closely related to cortical or striatal somatostatin (+) neurons. However, some level of LGE-specific transcripts were observed (FIG. 14). Reproducibility was strong within biological replicates as demonstrated by hierearchical clustering (FIG. 13A) and validated by qPCR (FIG. 13F). Protein expression assessed by mass spectrometry highlighted expression of synaptic and GABAergic machinery, neuronal markers, and downregulation of cell cycle components within iPSC-derived GABAergic neurons (FIG. 5F, FIG. 15A-E). Reactome pathway analysis highlighted a strong enrichment for GABA synthesis and release machinery (FIG. 15C, D). Moreover, hiPSC-derived GABAergic neurons were not proliferating (not shown) and downregulated expression of Ki67 (FIG. 5G) but did express GAD65 (FIG. 15E), as well as the glycinergic neuron-specific marker glycine transporter GlyT2 (FIG. 15E), the GABA transporter required for GABA release (Vesicular GABA/Glycine transporter VGAT) (FIG. 5H), and produced GABA (FIG. 51).

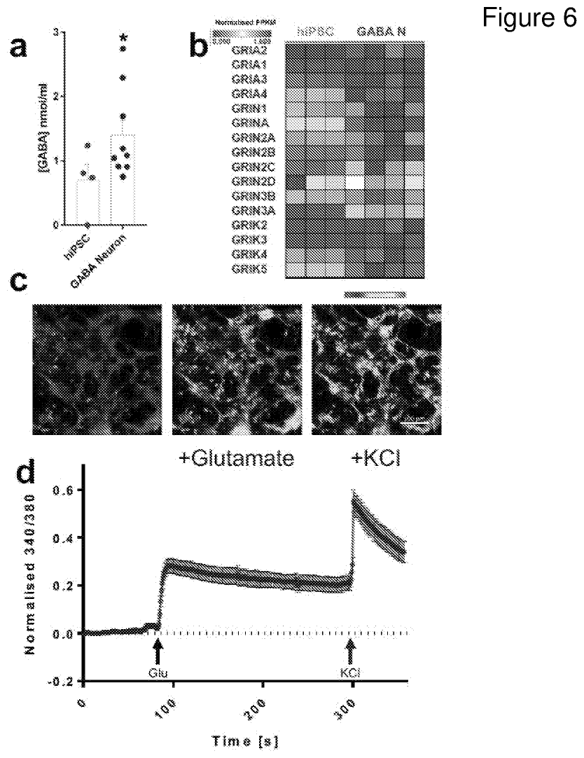

Since the iPSC-derived GABAergic (iGABAergic) neurons express GABAergic markers and machinery, the inventors evaluated GABA secretion and confirmed iGABAergic neurons secrete GABA by ELISA (FIG. 6A). Moreover, iGABAergic neurons express subunits of kainate, NMDA and AMPA glutamate receptor subclasses (FIG. 6B). To test if the cells were responsive, we stimulated the cells with glutamate and performed calcium imaging. We observed strong calcium transients and the majority of cells responded to both glutamate and potassium chloride (FIG. 6C, D).

TABLE 1
RNASeq of GABAergic neurons compared to hiPSC and hiPSC derived
Sensory neurons (FIGS. 5 and 13) (Normalised LogFPKM RNASEQ):

| | Ipsc1 | Ipsc2 | Ipsc3 | Gaba1 | Gaba2 | Gaba3 | Gaba4 |
|---|---|---|---|---|---|---|---|
| GAD1 | 0.0062 | 0.0052 | 0.0041 | 0.9225 | 1.0000 | 0.8579 | 0.9173 |
| GAD2 | 0.0109 | 0.0103 | 0.0102 | 0.8929 | 1.0000 | 0.9287 | 0.8323 |
| VGAT | 0.0062 | 0.0036 | 0.0049 | 0.8480 | 1.0000 | 0.9778 | 0.8731 |
| ABAT | 0.0210 | 0.0198 | 0.0215 | 0.9626 | 1.0000 | 0.9659 | 0.9524 |
| SST | 0.0046 | 0.0030 | 0.0099 | 0.8313 | 0.9168 | 1.0000 | 0.8647 |
| CALB1 | 0.7018 | 0.9980 | 1.0000 | 0.4662 | 0.4248 | 0.4277 | 0.4067 |
| CALR | 0.9632 | 0.9969 | 1.0000 | 0.3703 | 0.3119 | 0.3573 | 0.3597 |
| PVALB | 1.0000 | 0.7349 | 0.6202 | 0.0473 | 0.0270 | 0.0646 | 0.1018 |
| OCT4 | 1.0000 | 0.9626 | 0.9680 | 0.0008 | 0.0009 | 0.0010 | 0.0010 |
| NANOG | 1.0000 | 0.9247 | 0.9359 | 0.0011 | 0.0010 | 0.0010 | 0.0017 |
| MKI67 | 0.9975 | 1.0000 | 0.9139 | 0.2977 | 0.1645 | 0.1838 | 0.2991 |
| MCM2 | 0.8144 | 0.9647 | 1.0000 | 0.1368 | 0.1014 | 0.1192 | 0.1421 |
| SOX6 | 0.0144 | 0.0107 | 0.0073 | 0.9952 | 1.0000 | 0.9802 | 0.9877 |
| OLIG2 | 0.0159 | 0.0094 | 0.0050 | 1.0000 | 0.7031 | 0.9648 | 0.9061 |
| GFAP | 0.9941 | 0.9956 | 1.0000 | 0.2687 | 0.4419 | 0.1056 | 0.3815 |
| ALPL | 0.9829 | 0.9580 | 1.0000 | 0.0229 | 0.0204 | 0.0254 | 0.0212 |
| CCNB1 | 0.8315 | 0.9645 | 1.0000 | 0.1293 | 0.0809 | 0.0945 | 0.1232 |
| CDK1 | 0.9135 | 1.0000 | 0.9956 | 0.1547 | 0.0844 | 0.1063 | 0.1459 |
| CDK4 | 0.9624 | 1.0000 | 0.9944 | 0.4167 | 0.3615 | 0.4019 | 0.3895 |
| CSPG4 | 1.0000 | 0.7079 | 0.7259 | 0.0997 | 0.1274 | 0.1321 | 0.1864 |
| GAP43 | 0.0372 | 0.0331 | 0.0338 | 1.0000 | 0.9834 | 0.9717 | 0.9436 |
| GRIA1 | 0.0416 | 0.0385 | 0.0378 | 1.0000 | 0.9856 | 0.9809 | 0.9875 |
| GRIA2 | 0.0012 | 0.0022 | 0.0015 | 0.9312 | 1.0000 | 0.8349 | 0.9170 |
| GRIA3 | 0.1286 | 0.1014 | 0.0858 | 0.9762 | 1.0000 | 0.9166 | 0.9749 |
| GRIA4 | 0.3221 | 0.3273 | 0.2994 | 1.0000 | 0.9255 | 0.9062 | 0.9689 |
| GRIK2 | 0.0245 | 0.0302 | 0.0306 | 0.9788 | 0.9752 | 0.8922 | 1.0000 |
| GRIK3 | 0.0266 | 0.0327 | 0.0310 | 0.9830 | 0.9633 | 1.0000 | 0.9453 |
| GRIK4 | 0.2743 | 0.1993 | 0.2325 | 0.9250 | 0.8902 | 1.0000 | 0.9107 |
| GRIK5 | 0.3829 | 0.3570 | 0.3564 | 0.8630 | 1.0000 | 0.9251 | 0.8878 |
| GRIN2A | 0.2530 | 0.2720 | 0.2806 | 0.7727 | 0.8967 | 1.0000 | 0.7745 |
| GRIN2B | 0.1421 | 0.1261 | 0.1175 | 0.8496 | 1.0000 | 0.9787 | 0.9673 |
| GRIN2C | 0.1165 | 0.0930 | 0.1206 | 0.3600 | 1.0000 | 0.7621 | 0.3525 |
| GRIN2D | 1.0000 | 0.4239 | 0.3901 | 0.4971 | 0.7355 | 0.7107 | 0.5717 |
| GRIN3A | 0.0400 | 0.0586 | 0.0589 | 0.8489 | 0.9865 | 1.0000 | 0.8753 |
| GRIN3B | 0.3488 | 0.2929 | 0.3039 | 0.8225 | 1.0000 | 0.9322 | 0.7871 |
| GRINA | 0.4277 | 0.4063 | 0.3922 | 0.9031 | 1.0000 | 0.9863 | 0.9606 |
| KLF4 | 0.5439 | 1.0000 | 0.9498 | 0.1553 | 0.1853 | 0.1904 | 0.1370 |
| MBP | 1.0000 | 0.7992 | 0.7960 | 0.5560 | 0.4729 | 0.6016 | 0.5167 |
| MOG | 1.0000 | 0.4458 | 0.6556 | 0.0175 | 0.0400 | 0.0573 | 0.0565 |
| OLIG1 | 0.1227 | 0.0168 | 0.0088 | 1.0000 | 0.8210 | 0.9017 | 0.7555 |
| OLIG3 | 0.1485 | 1.0000 | 0.8806 | 0.9536 | 0.4620 | 0.6937 | 0.5283 |
| PCNA | 0.8851 | 0.9898 | 1.0000 | 0.2110 | 0.1732 | 0.1890 | 0.1982 |
| PODXL | 0.8530 | 0.9863 | 1.0000 | 0.0519 | 0.0519 | 0.0530 | 0.0543 |

TABLE 2
Validation by qPCR of select GABA synthesis genes (FIG. 13) (qPCR Log2 Fold Change).

| | Brain | Spinal Cord | hiPSC1 | hiPSC2 | hiPSC3 | GABAN1 | GABAN2 | GABAN3 | SensN1 | SensN2 | SensN3 |
|---|---|---|---|---|---|---|---|---|---|---|---|
| GAD1 | 9.9 | 4.9 | 0.0 | −0.1 | 0.2 | 8.8 | 9.0 | 8.7 | 4.1 | 4.0 | 3.3 |
| GAD2 | 8.7 | 4.1 | 0.0 | 0.9 | 1.0 | 8.1 | 7.9 | 8.0 | −3.1 | −3.2 | −2.9 |
| Nkx2.1 | 3.7 | 1.8 | 0.0 | 2.2 | 0.1 | 13.7 | 13.6 | 13.5 | 5.0 | 4.3 | 2.1 |
| vGAT | 9.4 | 5.7 | 0.0 | 0.5 | −0.3 | 9.7 | 9.0 | 9.7 | 4.2 | 3.5 | 3.0 |
| Lhx6 | 3.9 | 2.0 | 0.0 | −0.3 | −1.5 | 3.5 | 4.9 | 4.9 | −0.1 | −0.8 | −2.1 |
| Sox6 | 9.0 | 8.7 | 0.0 | 0.0 | −1.1 | 7.7 | 8.4 | 9.3 | 5.8 | 7.6 | 7.2 |
| Caltrenin | 7.5 | 5.6 | 0.0 | 1.5 | 6.2 | 13.1 | 13.4 | 11.9 | 11.7 | 8.8 | 0.0 |
| Calbindin | 1.6 | 0.5 | 0.0 | −0.4 | 4.9 | 12.5 | 11.6 | 11.4 | 15.1 | 9.6 | 11.9 |
| SST | 11.3 | 8.0 | 0.0 | −0.2 | 4.8 | 19.4 | 19.1 | 18.3 | 24.3 | 18.0 | 20.8 |
| Dcx | 3.9 | 2.1 | 0.0 | −.4 | 5.2 | 20.4 | 18.6 | 18.7 | 19.9 | 14.6 | 17.9 |
| Pax6 | 9.2 | 8.7 | 0.0 | 1.1 | 4.9 | 14.5 | 12.4 | 11.6 | 26.1 | 19.8 | 22.4 |
| b3-Tub | 12.4 | 11.4 | 0.0 | −0.2 | 7.0 | 26.9 | 26.3 | 25.5 | 28.3 | 21.1 | 23.8 |
| Oligo2 | 10.2 | 8.9 | 0.0 | 0.5 | −1.2 | 6.4 | 5.6 | 5.0 | 3.9 | 3.2 | 2.5 |
| GFAP | 14.5 | 17.2 | 0.0 | 0.0 | 2.9 | 0.0 | −2.5 | 0.0 | 0.0 | 0.0 | 0.0 |
| vGlut1 | 10.6 | 0.8 | 0.0 | −0.6 | −1.0 | −1.8 | −1.8 | −2.9 | 1.9 | 1.2 | 1.8 |

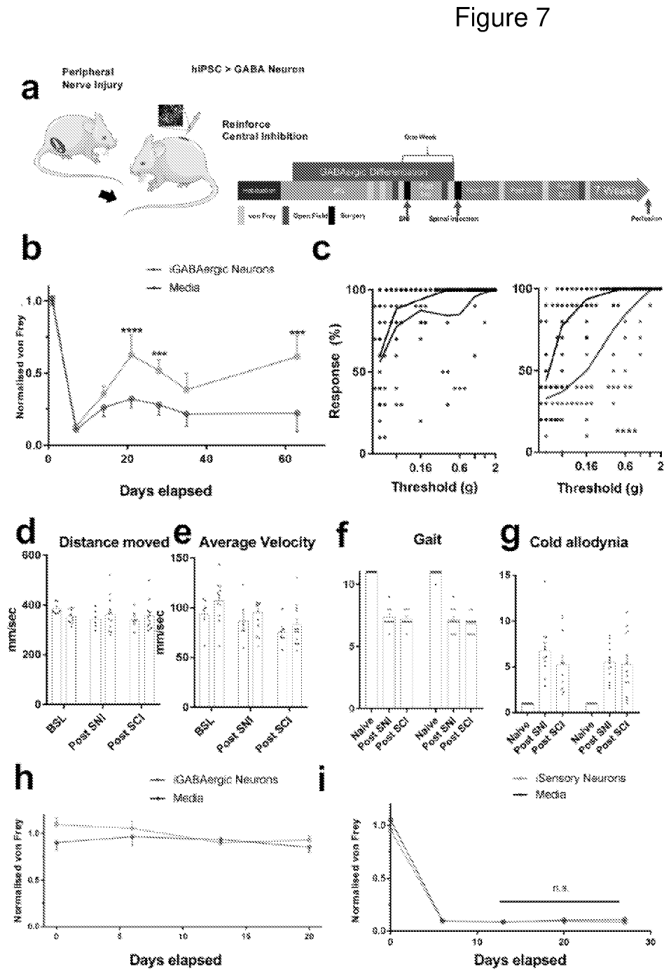

To assess the translational potential for iGABAergic neurons to treat pain disease, the inventors performed the spared nerve injury (SNI) model of neuropathic pain (Decosterd and Woolf, 2000) and then transplanted iGABAergic neurons into the ipsilateral dorsal horn of the spinal cord (lumbar enlargement) of injured mice (FIG. 6A). To avoid a xenograft response, the inventors used immune compromised (NOD^{SCID}) recipients. As expected SNI caused tactile and cold allodynia in NOD scid mice within 6 days; assessed by a von Frey light touch assay, and acetone response respectively. From two weeks after the transplant potent analgesia which lasted as long as two months (the endpoint for the study) was observed, manifesting as a reduction in tactile allodynia and a shift in the stimulus response curves (FIG. 7B-C).

Since GABAergic neurons are also involved in motor circuitry the inventors assessed motor behaviour using the open field test. No difference was observed between groups in either the maximal velocity or the average velocity of movement (FIG. 7D-E). Furthermore, the gait of the mice was assessed using a modified Basso Mouse Scale. SNI had an effect on gait as expected due to the damage to the mixed motor/sensory sciatic nerve. However, no gross change in gait between groups following transplantation was observed, together suggesting no major interference with motor behaviour (FIG. 7F). Due to close anatomical proximity, GABAergic neurons implanted into the lumbar dorsal horn have the potential to impact upon autonomic nervous system nuclei located in the spinal cord. However, no issues with bladder or bowel movements in the mice were observed.

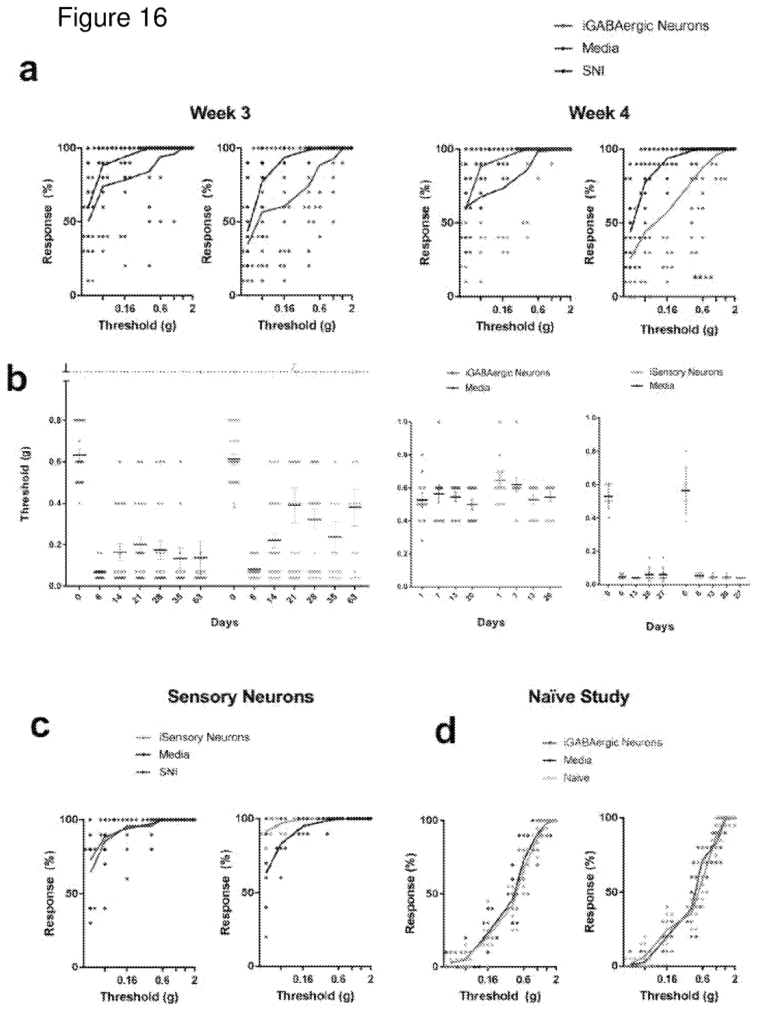

To further confirm the observed effects were unrelated to motor behaviour, iGABAergic neurons were transplanted into naïve mice. No change was seen in stimulus response and withdrawal threshold suggesting a nerve injury is required for the reduction in tactile sensitivity (FIG. 7H and FIG. 16). These results also confirmed the transplants do not simply increase the tonic threshold at which any stimulus is perceived (FIG. 16B). Finally, the inventors confirmed the specificity of treatment, since transplantation of hiPSC derived sensory neurons (iSensory neurons) into the dorsal horn following nerve injury was unable to elicit analgesia (FIG. 71 and FIG. 16C). Taken together, enhancing inhibition using mature iGABAergic neuron transplants promote long-term analgesia from peripherally induced neuropathic pain.

To assess the potential for neurons to engraft in the spinal cord, cell survival, integration and potential for synaptic integration was assessed (FIG. 8). Three weeks post transplantation we identified hiPSC-derived GABAergic neurons in the dorsal horn laminae I (CGRP+) and II (IB4+) as well as other dorsal laminae through radial migration (FIG. 8). Initially, iGABAergic neurons did not migrate to ventral or contralateral spinal cord (FIG. 8), retained neuronal and GABAergic marker expression and showed early evidence of synaptic integration potential (FIG. 8). However, after 10 weeks substantial migration was observed. Transplanted cells retained their neuronal identity as assessed by their co-expression of human NCAM1, MAP2, TUBB3 and expressed NeuN suggesting terminal differentiation (FIG. 9C-E). iGABAergic neurons were immunoreactive to GABA and retained VGAT, GAD65/67 and synapsin expression indicating transplanted iGABAergic neurons retain the ability to synthetise, package and release GABA (FIG. 9F-H). Additionally, it was found that mouse presynaptic densities (marked by a mouse-specific antibody targeting Bassoon and co-localising with presynaptic protein RIM2) were in direct apposition to iGABAergic neurons (marked by a human specific cytoplasmic antibody, HuCytoplasm), suggesting the potential to form synapses between transplant and recipient tissue (FIG. 91). iGABAergic neurons expressed critical presynaptic proteins, liprin and were immunoreactive to a pan-voltage gated calcium channel antibody (FIG. 9K-L). Finally, the apposition of the inhibitory post synaptic marker gephyrin with human synapsin we observed, suggesting the presence of inhibitory synapses (FIG. 9M-N). Of note, the transplanted inhibitory neurons were predominately somatostatin or parvalbumin subclasses (FIG. 9O-P). Importantly, no morphological evidence of teratoma or other related abnormalities was observed and no evidence of proliferation of iGABAergic neurons could be identified (the cells did not express active cell cycle protein Ki67) (FIG. 9R-S), which highlights the safety of the procedure.

### Example 6. Enhanced Differentiation of hiPSC-GABA Neurons to Relieve Pain in Neuropathic Mice

iGABAergic neurons were prepared according to an adaptation of the method described in Example 5. During the GABAergic differentiation protocol, hiPSCs were exposed to BMP4, during week 3 only (i.e. about DIV14 to about DIV21), week 4 only (i.e. about DIV21 to about DIV28) or during week 3 and week 4 (i.e. about DIV14 to about DIV28). Following differentiation (i.e. DIV28) the iGABAergic neurons were assessed for expression of somatostatin (SST) and Parvalbumin (PVALB). BMP4 treated cells were then be tested for their ability to reverse neuropathic pain and compared to non-treated iGABAergic neurons using the SNI model as hereinbefore described.

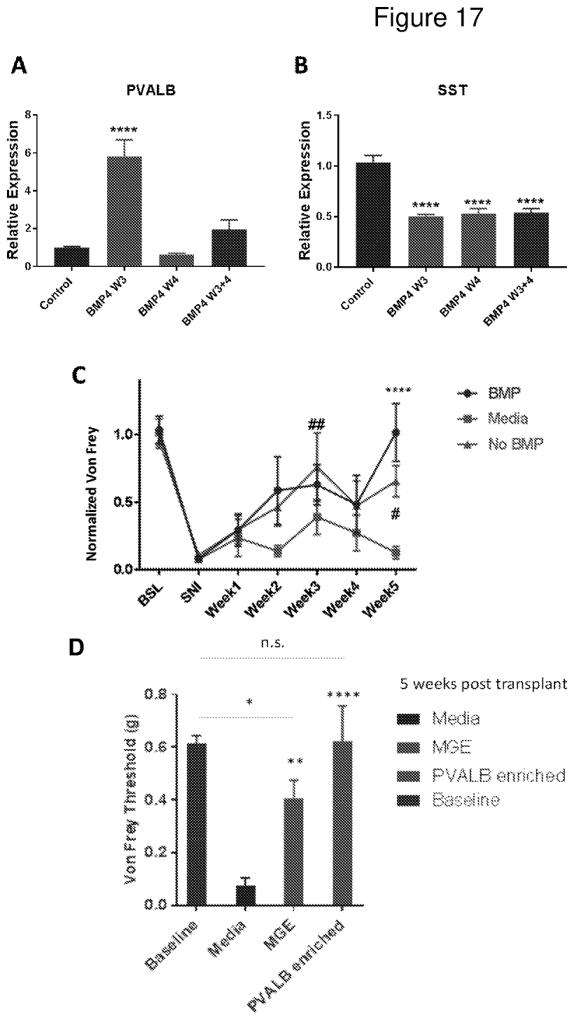

Surprisingly, it was found that exposure of hiPSCs to BMP4 during week 3, but not week 4 or week 3 and 4, leads to a 6-fold increase of PAVB expression. Exposure of hiPSCs during either or both of weeks 3 and 4 in the differentiation protocol led to a 50% reduction of SST expression shown by qPCR (FIG. 17A,B).

Surprisingly, it was also found that when iGABAergic neurons that were exposed to BMP4 during week 3 of the differentiation protocol (Parvalbumin enriched) were transplanted into SNI mice, a significantly more potent long-term analgesic response was observed at 5 weeks post injury compared to when SNI mice were treated with iGABAergic neurons that were not exposed to BMP4 (FIG. 17C). Indeed, animals treated with Parvalbumin enriched iGABAergic neurons showed no significant difference compared to their respective baseline control 5 weeks post-transplant (i.e. pain is fully back to normal in SNI animals) (FIG. 17C).

Exploiting the core role for central disinhibition as a key mechanism in neuropathic pain, the inventors show hiPSC-derived GABAergic cultures can be transplanted into the spinal cord of neuropathic mammals to promote long lasting disease relief. In the studies described herein there no obvious behavioural or physiological adverse response to hiPSC-derived GABAergic neuron transplantation were observed. Moreover, GABAergic neurons were not dividing, displayed downregulated cell cycle and pluripotency markers, and did not form tumours or teratomas when transplanted into recipient animals, highlighting not only the efficacy but the safety of the compositions and methods of the present invention.

Together, these data show that central inhibition is a core primordial pathology critical for some forms of neuropathic pain, and reinforcing central inhibitory tone via anti-neuropathic GABAergic transplants can promote long term and potentially curative relief from neuropathic pain. Existing analgesics work for hours and some have serious adverse effects. This therapy represents the possibility of a single procedure that provides longer lasting analgesia and effectively alleviates neuropathic pain without side effects.

1. A transplant composition for administration to a mammal, said transplant composition comprising a population of GABAergic neurons, a GFRalpha agonist and at least one cell death inhibitor, wherein said GABAergic neurons are generated by differentiating pluripotent stem cells, or multipotent stem cells or progenitor cells in vitro under conditions to permit the cells to obtain a GABAergic neuronal phenotype and to produce GABA.

2. The transplant composition according to claim 1, comprising an apoptosis inhibitor and a necrosis inhibitor.

3. The transplant composition according to claim 1 or 2, wherein the GABAergic neurons are generated by culturing said pluripotent stem cells, or multipotent stem or progenitor cells in the presence of:
i. at least two SMAD inhibitors from about day 0 to about day 7;
ii. an activator of sonic hedgehog pathway from about day 0 to about day 21;
iii. a wnt inhibitor from about day 0 to about day 14;
iv. a BMP inhibitor from about day 7 to about day 14;
v. a GABAergic speciation factor from about day 7 to about day 21; and
vi. a combination of neuronal maturation growth factors comprising BDNF, GDNF and a gamma secretase inhibitor from about day 21 to about day 27.

4. The transplant composition according to claim 3, wherein said pluripotent stem cells, or multipotent stem or progenitor cells are cultured in the presence of one or more agents which activate BMP signalling during the period from about day 14 to about day 21.

5. The transplant composition according to claim 4, wherein said one or more agents which activate BMP signalling comprises BMP4.

6. The transplant composition according to any one of the preceding claims, wherein the pluripotent stem cells, or multipotent stem or progenitor cells are obtained from said mammal.

7. The transplant composition according to any one of the preceding claims, wherein the GABAergic neurons are generated from pluripotent stem cells.

8. The transplant composition according to claim 7, wherein the pluripotent stem cells are iPSCs.

9. The transplant composition according to any one of the preceding claims, wherein the GFRalpha agonist is selected from the group consisting of glial cell-derived neurotrophic factor (GDNF), Neurturin (NRTN), Artemin (ARTN) and Persephin (PSPN), Brain-derived neurotrophic factor (BDNF), NGF, GDNF receptor endogenous agonists, BT18, BT13, NT-3, NT-4, CNTF, GFRalphal Agonists, XIB4035, Trk activator, TrkA Agonists, Gamobogic Amine, Amitriptyline, TrkB agonists, N-Acetylserotonin, Amitriptyline, BNN-20BNN-27, Deoxygedunin, 7,8-Dihydroxyflavone, 4′-Dimethylamino-7,8-dihydroxyflavone, Diosmetin, HIOC, LM22A-4, Neurotrophin-3, Neurotrophin-4, Norwogonin, R7 (drug), and 7,8,3′-Trihydroxyflavone.

10. The transplant composition according to claim 9, wherein the GFRalpha agonist is GDNF.

11. The transplant composition according to any one of claims 2-10, wherein the apoptosis inhibitor is selected from one or more of, a caspase inhibitor selected from the group consisting of Boc-Asp(OMe) fluoromethyl ketone IDN-8066, 7053, 7436 1965 6556 M867 IDN-5370 IDN-7866 pralnacasan z-Vad-FMK, YVAD-FMK, c-DEVD-CHO, Ac-YVAD-CHO, Ac-DVAD-FMK Q-Vd-OPh, CrmA (cowpox virus protein), p35 (Bacoluvirus protein), Z-ATAD-FMK, INF-4E, Z-DQMD-FMK, Az 10417808, Z-LEED-FMK, ZVDK-FMK, z-IETD-FMK, INf-39, Belnacasan, Ac-DEVD-CHO, and Emricasan; or a an inhibitor of a caspase activator selected from the group consisting of Calpain inhibitor 1, Calpeptin, E64, MDL28170, MG101, Acetyl-Calpastatin, and PD 150606.

12. The transplant composition according to claim 10, wherein the apoptosis inhibitor is a broad-spectrum caspase inhibitor.

13. The transplant composition according to claim 12, wherein the apoptosis inhibitor is Boc-Asp(OMe) fluoromethyl ketone.

14. The transplant composition according to any one of claims 2-13, wherein the necrosis inhibitor is selected from the group consisting of MS-1, IM-54, GSK-872, 7-Cl-O-Nec1, Necrostatin-1, and Necrosulfonamide.

15. The transplant composition according to claim 14, wherein the necrosis inhibitor is a MLKL inhibitor.

16. The transplant composition according to claim 15, wherein the necrosis inhibitor is necrosulfonamide.

17. A method for producing a population of GABAergic neurons comprising: culturing pluripotent stem cells, or multipotent stem cells or progenitor cells in vitro in the presence of i. at least two SMAD inhibitors from about day 0 to about day 7;
ii. an activator of sonic hedgehog pathway from about day 0 to about day 21;
iii. a wnt inhibitor from about day 0 to about day 14;
iv. a BMP inhibitor from about day 7 to about day 14;
v. a GABAergic speciation factor from about day 7 to about day 21; and
vi. a combination of neuronal maturation growth factors comprising BDNF, GDNF and a gamma secretase inhibitor from about day 21 to about day 27 such that the cells obtain a GABAergic neuronal phenotype and produce GABA.

18. The method according to claim 17, wherein said pluripotent stem cells, or multipotent stem or progenitor cells are cultured in the presence of one or more agents which activate BMP signalling during the period from about day 14 to about day 21.

19. The method according to claim 18, wherein said one or more agents which activate BMP signalling comprises BMP4.

20. The transplant composition according to any one of claims 1-16, or the method of any one of claims 17-19, wherein said GABAergic neurons are post-mitotic.

21. The transplant composition according to any one of claim 1-16 or 20, or the method of any one of claims 17-20, wherein said GABAergic neurons express transcripts for Nkx2.1, vGAT, GAD65, GAD67.

22. The transplant composition according to any one of claims 1-16 or 20-21, or the method of any one of claims 17-21, wherein said GABAergic neurons express GAD65/67, GlyT2 and VGAT.

23. The transplant composition according to any one of claims 1-16 or 20-22, or the method of any one of claims 17-22, wherein at least 95% of said population of GABAergic neurons express GAD65.

24. The transplant composition according to any one of claims 1-16 or 20-23, or the method of any one of claims 17-23, wherein at least 95% of said population of GABAergic neurons express VGAT.

25. The transplant composition according to any one of claims 1-16 or 20-24, or the method of any one of claims 17-24, wherein said GABAergic neurons are capable of secreting GABA in vivo.

26. The transplant composition according to any one of claims 3-16 or 20-25, or the method of any one of claims 17-25, wherein said at least two SMAD inhibitors are selected from the group consisting of Hesperetin, SB431542, SB525334, Galunisertib, GW788388, LY2109761, SB505124, LDN-193189, LDN-193189 HCl, RepSox, A 83-01, DMH1, LDN-212854, ITD 1, LY364947, SD-208, EW-7197, ML347, K02288, A 77-01, SIS3, LDN-214117, R-268712, Pirfenidone, Noggin, Chordin, Gremlin, DAN proteins, and GDF3.

27. The transplant composition or method according to claim 26, wherein said at least two SMAD inhibitors are LDN193189 and SB431542.

28. The transplant composition according to any one of claims 3-16 or 20-27, or the method of any one of claims 17-27, wherein said activator of sonic hedgehog signaling is selected from the group consisting of Sonic Hedgehog, GSA10, Purmorphamine, SAG, and SAG dihydrochloride.

29. The transplant composition or method according to claim 28, wherein said activator of sonic hedgehog signaling is SAG.

30. The transplant composition according to any one of claims 3-16 or 20-29, or the method of any one of claims 17-29, wherein said wnt inhibitor is selected from the group consisting of ICG-001, Salinomycin, IWR-1, Wnt-059, ETC-159, iCRT3, IWP2, IWP-4, Pyrvinium Pamoate, iCRT14, FH535, CCT251545, KYA1797K, Wogonin, NCB-0846, Hexachrorophene, PNU-74654, Ky0211, Triptonide, IWP12, Axin, GSK, WAY316606, Shizokaol D, BC2059, PKF115-584, ICG-01, Quercetin, DCA, LY2090314, CHIR99021, SB-216763, NSC668036, QS11, G007-LK, and G244LM.

31. The transplant composition or method according to claim 30, wherein said wnt inhibitor is IWP2.

32. The transplant composition according to any one of claims 3-16 or 20-31, or the method of any one of claims 17-31, wherein said BMP inhibitor is selected from the group consisting of Hesperetin, SB431542, SB525334, Galunisertib, GW788388, LY2109761, SB505124, LDN-193189, LDN-193189 HCl, RepSox, A 83-01, DMH1, LDN-212854, ITD 1, LY364947, SD-208, EW-7197, ML347, K02288, A 77-01, SIS3, LDN-214117, R-268712, Pirfenidone, Noggin, Chordin, Gremlin, DAN proteins, and GDF3.

33. The transplant composition or method according to claim 32, wherein said BMP inhibitor is LDN-193189.

34. The transplant composition according to any one of claims 3-16 or 20-33, or the method of any one of claims 17-33, wherein said GABAergic speciation factor is selected from the group consisting of Fibroblasts Growth Factors.

35. The transplant composition or method according to claim 34, wherein said GABAergic speciation factor is FGF8.

36. The transplant composition according to any one of claims 3-16 or 20-35, or the method of any one of claims 17-35, wherein said gamma secretase inhibitor is selected from the group consisting of DAPT, RO4929097, Semagecestat, Avagacestat, Dibenzazipine, Ly411575, IMR-1, L-685,458, FLI-06, Crenigacestat, Nirogacestat, MK-0752, Begacestat, BMS299897, Compound W, DBZ, Flurizan, JLK6, MRK560, and PF3084014 hydrobromide.

37. The transplant composition or method according to claim 36, wherein said gamma secretase inhibitor is DAPT.

38. The transplant composition according to any one of claims 3-16 or 20-37, or the method of any one of claims 17-29, wherein said pluripotent stem cells, or multipotent stem cells or progenitor cells are cultured in the presence of a Rock inhibitor from day 0 for a period of about 24 h.

39. A population of cells produced according to the method of any one of claims 17-38.

40. A method of restoring or reinforcing central inhibition in the nervous system of a mammal comprising administering to the mammal a transplant composition according to any one of claim 1-16 or 20-38, or a therapeutically effective amount of a population of cells produced according to the method of any one of claims 17-38.

41. A method of treating a neurological condition, disease or disorder, or allodynia in a mammal comprising administering to the mammal a transplant composition according to any one of claim 1-16 or 20-38, or a therapeutically effective amount of a population of cells produced according to the method of any one of claims 17-38.

42. A transplant composition according to any one of claim 1-16 or 20-38, or a therapeutically effective amount of a population of cells produced according to the method of any one of claims 17-38 for the treatment of inadequate inhibitory interneuron activity or increased excitatory neuron function in a subject.

43. A transplant composition according to any one of claim 1-16 or 20-38, or a therapeutically effective amount of a population of cells produced according to the method of any one of claims 17-38 for the treatment of a neurological condition, disease or disorder, or allodynia in a subject.

44. Use of a transplant composition according to any one of claim 1-16 or 20-38, or a therapeutically effective amount of a population of cells produced according to the method of any one of claims 17-38 for the manufacture of a medicament for the treatment of a neurological condition, disease or disorder, or allodynia in a subject.

45. The method according to claim 41, the transplant composition according to any one of claim 1-16 or 20-38 or therapeutically effective amount of a population of cells produced according to the method of any one of claims 17-38 for the use of claim 43, or the use according to claim 44, wherein the neurological condition, disease or disorder is selected from a neurodegenerative disease, neurological injury, or neuropathic pain.

46. The method according to claim 41, the transplant composition according to any one of claim 1-16 or 20-36 or therapeutically effective amount of a population of cells produced according to the method of any one of claims 17-38 for the use of claim 43, or the use according to claim 44, wherein said neurological condition, disease or disorder is selected from the group consisting of: Chronic Neuropathic pain, Chronic Inflammatory Pain, Chronic dysfunctional Pain, Epilepsy, Motor neuron disease (ALS, SMA), Parkinson's Disease, Alzheimer's Disease, Stroke, Multiple Sclerosis, Tauopathies (Progressive Supranuclear Palsy, Pick's disease, Cortical Basal Degeneration (CBD), Frontotemporal lobe dementia, FTLD with ALS), Huntington's disease, Alcohol withdrawal and Alcoholism, Diabetes induced brain damage, Head injury, Migraine, Headache, Cluster Headache, Spinal Cord Injury, Ischaemic Damage, Chemotherapy induced pain and chemotherapy induced neuropathy, Schizophrenia, Chronic Depression, Tardive Dyskinesia, Bipolar Disorder, and Neuropathies.

47. The method according to claim 45, the transplant composition according to any one of claim 1-16 or 20-38 or therapeutically effective amount of a population of cells produced according to the method of any one of claims 17-38 for the use of claim 45, or the use according to claim 45, wherein said neuropathic pain is associated with sciatica, back pain, cancer pain, diabetic pain, accidental injury, spinal cord injury, peripheral nerve injury.

48. The method according to any one of claims 40, 41 or 45-47, the transplant composition according to any one of claim 1-16 or 20-38 or therapeutically effective amount of a population of cells produced according to the method of any one of claims 17-38 for the use of claims 42, 43, or 45-47, or the use according to any one of claims 44-47, wherein said transplant composition, said therapeutically effective amount of a population of cells or said medicament is administered or is formulated for administration to the central nervous system of the mammal.

49. The method according to any one of claims 40, 41 or 45-47, the transplant composition according to any one of claim 1-16 or 20-38 or therapeutically effective amount of a population of cells produced according to the method of any one of claims 17-38 for the use of claims 42, 43, or 45-47, or the use according to any one of claims 44-47, wherein said transplant composition, said therapeutically effective amount of a population of cells or said medicament is injected, or is formulated for injection, into the spinal cord of said mammal.

50. The method according to claim 49, the transplant composition according to any one of claim 1-16 or 20-38 or therapeutically effective amount of a population of cells produced according to the method of any one of claims 17-38 for the use of claim 49, or the use according to claim 49, wherein said, wherein said injecting is stereotactic injection.

51. The method according to any one of claim 40, 41 or 45-50, the transplant composition according to any one of claim 1-16 or 20-38 or therapeutically effective amount of a population of cells produced according to the method of any one of claims 17-38 for the use of claim 42, 43, or 45-50, or the use according to any one of claims 44-50, wherein the mammal is a human.

52. A method of delivering GABAergic neurons to a subject in need thereof, said method comprising the steps of:
a) obtaining a biopsy from said subject and isolating cells from said biopsy;
b) generating iPSCs from cells isolated in step a);
c) culturing the iPSCs generated in step b) under conditions to differentiate said iPSCs into GABAergic neurons, wherein said GABAergic neurons express a GABAergic neuronal phenotype and produce GABA;
d) preparing a transplant composition suitable for injection to a said subject, said transplant composition comprising the GABAergic neurons generated in step c), a GFRalpha agonist, an apoptosis inhibitor, a necrosis inhibitor, and a pharmaceutically acceptable carrier;
e) administering the transplant composition prepared in step d) to said subject.
